SEQUENCE LISTING (52 sequences)

SEQ ID NO: 1 - DNA, 216 bp, Artificial
  synthetic oligonucleotide encoding for the leader sequence, the transmembrane and cytoplasmic tail of Mus musculus LAMP-1, containing Nhe I site 3′ of the LAMP-1 leader sequence and Nde I site 5′ of the LAMP-1 transmembrane and cytop
ctcgagccaccatggccgcccccggcgcccggaggcccctgctcctgctgctgctggcaggccttgcacatggcgctagcgaattcccggggatccatatgttgatccccattgctgtgggcggtgccctggcagggctggtcctcatcgtcctcatcgcctacctcattggcaggaagaggagtcacgccggctatcagaccatctagcggccgc

SEQ ID NO: 2 - DNA, 234 bp, Artificial
  chimeric gene that encodes the Mus musculus LAMP-1 leader sequence,  the Blo t5 gene fragment for the H-2d-restricted Thepitope and the Mus musculus LAMP-1 transmembrane and cytoplasmic domain
atggccgcccccggcgcccggaggcccctgctcctgctgctgctggcaggccttgcacatggcgctagcgcagaattgcaagagaaaatcattcgagaacttgatgttgtttgcgccatgaatatgttgatccccattgctgtgggcggtgccctggcagggctggtcctcatcgtcctcattgcctacctcattggcaggaagaggagtcacgccggctatcagaccatctag

SEQ ID NO: 3 - DNA, 534 bp, Artificial
  chimeric gene that encodes the Mus musculus LAMP-1 leader sequence, the entire Blo t 5 gene product and the Mus musculus LAMP-1 transmembrane and cytoplasmic domain
atggccgcccccggcgcccggaggcccctgctcctgctgctgctggcaggccttgcacatggcgctagccaagagcacaagccaaagaaggatgatttccgaaacgaattcgatcacttgttgatcgaacaggcaaaccatgctatcgaaaagggagaacatcaattgctttacttgcaacaccaactcgacgaattgaatgaaaacaagagcaaggaattgcaagagaaaatcattcgagaacttgatgttgtttgcgccatgatcgaaggagcccaaggagctttggaacgtgaattgaagcgaactgatcttaacattttggaacgattcaactacgaagaggctcaaactctcagcaagatcttgcttaaggatttgaaggaaaccgaacaaaaagtgaaggatattcaaacccaaaatatgttgatccccattgctgtgggcggtgccctggcagggctggtcctcatcgtcctcatcgcctacctcattggcaggaagaggagtcacgccggctatcagaccatctag

SEQ ID NO: 4 - DNA, 420 bp, Artificial
  chimeric gene that encodes the Mus musculus LAMP-1 leader sequence and the entire Blo t 5 gene product
atggccgcccccggcgcccggaggcccctgctcctgctgctgctggcaggccttgcacatggcgctagccaagagcacaagccaaagaaggatgatttccgaaacgaattcgatcacttgttgatcgaacaggcaaaccatgctatcgaaaagggagaacatcaattgctttacttgcaacaccaactcgacgaattgaatgaaaacaagagcaaggaattgcaagagaaaatcattcgagaacttgatgttgtttgcgccatgatcgaaggagcccaaggagctttggaacgtgaattgaagcgaactgatcttaacattttggaacgattcaactacgaagaggctcaaactctcagcaagatcttgcttaaggatttgaaggaaaccgaacaaaaagtgaaggatattcaaacccaa

SEQ ID NO: 5 - DNA, 849 bp, Artificial
  chimeric gene that encodes the Mus musculus LAMP-1 leader sequence, the entire Der p 1 gene product and the Mus musculus LAMP-1 transmembrane and cytoplasmic domain
atggccgcccccggcgcccggaggcccctgctcctgctgctgctggcaggccttgcacatggcgctagcactaacgcctgcagtatcaatggaaatgctccagctgaaatcgatttgcgacaaatgcgaactgtcactcccattcgtatgcaaggaggctgtggttcatgttgggctttctctggtgttgccgcaactgaatcagcttatttggcttaccgtaatcaatcattggatcttgctgaacaagaattagtcgattgtgcttcccaacacggttgtcatggtgataccattccacgtggtattgaatacatccaacataatggtgtcgtccaagaaagctactatcgatacgttgcacgagaacaatcatgccgacgaccaaatgcacaacgtttcggtatctcaaactattgccaaatttacccaccaaatgtaaacaaaattcgtgaagctttggctcaaacccacagcgctattgccgtcattattggcatcaaagatttagacgcattccgtcattatgatggccgaacaatcattcaacgcgataatggttaccaaccaaactatcacgctgtcaacattgttggttacagtaacgcacaaggtgtcgattattggatcgtacgaaacagttgggataccaattggggtgataatggttacggttattttgctgccaacatcgatttgatgatgattgaagaatatccatatgttgtcattctcaatatgttgatccccattgctgtgggcggtgccctggcagggctggtcctcatcgtcctcatcgcctacctcattggcaggaagaggagtcacgccggctatcagaccatctag

SEQ ID NO: 6 - DNA, 879 bp, Artificial
  chimeric gene that encodes the Homo sapiens tissue plasminogen activator leader sequence, the entire Der p 1 gene product and the Mus musculus LAMP-1 transmembrane and cytoplasmic domain
atggatgcaatgaagagagggctctgctgtgtgctgctgctgtgtggagcagtcttcgtttcgcccagccaggttggtgtgcaggacccctgtgtcccgcccctcaccaacgcctgcagcatcaacggcaatgcccccgctgagattgatctgcgccagatgaggaccgtgactcccatccgcatgcaaggcggctgcgggtcttgttgggccttctcaggcgtggccgcgaccgagtctgcatacctcgcgtatcggaatcagagcctggacctcgctgagcaggagctcgttgactgcgcctcccaacacggatgtcatggggatacgattcccagaggtatcgaatacatccagcataatggcgtcgtgcaggaaagctattaccgatacgtagctagggagcagtcctgccgccgtcctaacgcccagcgcttcggcatttccaactattgccagatctacccccctaatgtgaacaagatcagggaggccctggcgcagacgcacagcgccatcgctgtcatcatcggaatcaaggatctggacgcattccggcactatgacgggcgcacaatcatccagcgcgacaacggataccagccaaactatcacgcggtcaacatcgtgggttactcgaacgcccagggggtggactactggatcgtgcggaacagttgggacaccaactggggcgacaacggctacggctactttgccgccaacatcgacctgatgatgatcgaagagtacccgtacgtggtgatcctgttgatccccattgctgtgggcggtgccctggcagggctggtcctcatcgtcctcattgcctacctcattggcaggaagaggagtcacgccggctatcagaccatctag

SEQ ID NO: 7 - protein, 26 aa, Rattus norvegicus LIMP II Leader peptide
MARCCFYTAGTLSLLLLVTSVTLLVA

SEQ ID NO: 8 - protein, 46 aa, Rattus norvegicus LIMP II Transmembrane cytoplasmic domain
LIVTNIPYIIMALGVFFGLIFTWLACRGQGSTDEGTADERAPLIRT

SEQ ID NO: 9 - protein, 26 aa, Homo sapiens LIMP II Leader peptide
MGRCCFYTAGTLSLLLLVTSVTLLVA

SEQ ID NO: 10 - protein, 46 aa, Homo sapiens LIMP II Transmembrane cytoplasmic domain
LIITNIPYIIMALGVFFGLVFTWLACKGQGSMDEGTADERAPLIRT

SEQ ID NO: 11 - protein, 26 aa, Mus musculus LIMP II Leader peptide
MGRCCFYTAGTLSLLLLVTSVTLLVA

SEQ ID NO: 12 - protein, 46 aa, Mus musculus LIMP II Transmembrane cytoplasmic domain
LVVTNIPYIIMALGVFFGLVFTWLACRGQGSMDEGTADERAPLIRT

SEQ ID NO: 13 - protein, 27 aa, Homo sapiens DEC-205 Leader peptide
MRTGWATPRRPAGLLMLLFWFFDLAEP

SEQ ID NO: 14 - protein, 56 aa, Homo sapiens DEC-205 Transmembrane cytoplasmic domain
YTAIAIIVATLSILVLMGGLIWFLFQRHRLHLAGFSSVRYAQGVNEDEIMLPSFHD

SEQ ID NO: 15 - protein, 27 aa, Mus musculus DEC-205 Leader peptide
MRTGRVTPGLAAGLLLLLLRSFGLVEP

SEQ ID NO: 16 - protein, 56 aa, Mus musculus DEC-205 Transmembrane cytoplasmic domain
YTGIAILFAVLCLLGLISLAIWFLLQRSHIRWTGFSSVRYEHGTNEDEVMLPSFHD

SEQ ID NO: 17 - protein, 41 aa, Homo sapiens P-selectin Leader peptide
MANCQIAILYQRFQRVVFGISQLLCFSALISELTNQKEVAA

SEQ ID NO: 18 - protein, 59 aa, Homo sapiens P-selectin Transmembrane cytoplasmic domain
LTYFGGAVASTIGLIMGGTLLALLRKRFRQKDDGKCPLNPHSHLGTYGVFTNAAFDPSP

SEQ ID NO: 19 - protein, 17 aa, Homo sapiens tyrosinase Leader peptide
MLLAVLYCLLWSFQTSA

SEQ ID NO: 20 - protein, 30 aa, Homo sapiens tyrosinase Transmembrane cytoplasmic domain
CRHKRKQLPEEKQPLLMEKEDYHSLYQSHL

SEQ ID NO: 21 - protein, 24 aa, Homo sapiens GLUT4 Leader peptide
MPSGFQQIGSEDGEPPQQRVTGTL

SEQ ID NO: 22 - protein, 43 aa, Homo sapiens GLUT4 Transmembrane Cytoplasmic domain
RVPETRGRTFDQISAAFHRTPSLLEQEVKPSTELEYLGPDEND

SEQ ID NO: 23 - protein, 21 aa, Rattus norvegicus endotubin Leader peptide
MCLPSCLLSIWVLFMAAQSLG

SEQ ID NO: 24 - protein, 66 aa, Rattus norvegicus endotubin Transmembrane Cytoplasmic domain
AAPVSVPVAVGGALLLFLLLLGLGGWHWLQKQHLPCQSTDAAASGFDNILFNADQVTLPESITSNP

SEQ ID NO: 25 - protein, 23 aa, Mus musculus LAMP-1 leader peptide
MAAPGARRPLLLLLLAGLAHGAS

SEQ ID NO: 26 - protein, 36 aa, Mus musculus LAMP-1 transmembrane and cytoplasmic domain
MLIPIAVGGALAGLVLIVLIAYLIGRKRSHAGYETI

SEQ ID NO: 27 - DNA, 78 bp, Rattus norvegicus nucleotide sequence encoding the LIMP II leader peptide
atggcccgatgctgcttctacacggcggggacactgtctctgctgctgctggtgaccagtgtcacgctgctagtggct

SEQ ID NO: 28 - DNA, 141 bp, Rattus norvegicus nucleotide sequence encoding the LIMP II Transmembrane cytoplasmic domain
ttgattgtcaccaacataccctacatcatcatggcactgggcgtgttctttggcttgattttcacgtggctggcgtgtcgaggacaggggtctacggatgagggaactgcagatgaaagggcacccctcatacggacctaa

SEQ ID NO: 29 - DNA, 78 bp, Homo sapiens nucleotide sequence encoding the LIMP II Leader peptide
atgggccgatgctgcttctacacggcggggacgttgtccctgctcctgctggtgaccagcgtcacgctgctggtggcc

SEQ ID NO: 30 - DNA, 141 bp, Homo sapiens nucleotide sequence encoding the LIMP II Transmembrane cytoplasmic domain
ttgatcatcaccaacataccctacatcatcatggcgctgggtgtgttctttggtttggtttttacctggcttgcatgcaaaggacagggatccatggatgagggaacagcggatgaaagagcacccctcattcgaacctaa

SEQ ID NO: 31 - DNA, 78 bp, Mus musculus nucleotide sequence encoding the LIMP II Leader peptide
atgggcagatgctgcttctacacggcggggacgctgtctctgctgctgctggtgaccagcgtcacgctgctagtggct

SEQ ID NO: 32 - DNA, 141 bp, Mus musculus nucleotide sequence encoding the LIMP II Transmembrane cytoplasmic domain
ttggttgtcaccaacataccctacatcattatggcactgggtgtgttctttggcttggttttcacgtggctggcgtgtcgaggacaggggtctatggatgagggaactgcagatgaaagagcacccctcatacgaacctaa

SEQ ID NO: 33 - DNA, 81 bp, Homo sapiens nucleotide sequence encoding the DEC-205 Leader peptide
atgaggacaggctgggcgacccctcgccgcccggcggggctcctcatgctgctcttctggttcttcgatctcgcggagccc

SEQ ID NO: 34 - DNA, 171 bp, Homo sapiens nucleotide sequence encoding the DEC-205 Transmembrane cytoplasmic domain
tacacagcaatagctatcatagttgccacactaagtatcttagttctcatgggcggactgatttggttcctcttccaaaggcaccgtttgcacctggcgggtttctcatcagttcgatatgcacaaggagtgaatgaagatgagattatgcttccttctttccatgactaa

SEQ ID NO: 35 - DNA, 81 bp, Mus musculus nucleotide sequence encoding the DEC-205 leader peptide
atgcggacgggccgggtgaccccgggcctggcggcggggctactcctgctgttgctgcggtccttcgggcttgtggagcct

SEQ ID NO: 36 - DNA, 171 bp, Mouse nucleotide sequence encoding the DEC-205 Transmembrane cytoplasmic domain
tacacaggcatagccatcctgtttgccgtgctgtgcctcttagggctcatcagcttggcgatttggttcctcttgcaacgatcccatatccgctggaccggcttctcctcggttcggtatgaacatggaaccaacgaagacgaggtgatgctcccttctttccacgactaa

SEQ ID NO: 37 - DNA, 123 bp, Homo sapiens nucleotide sequence encoding the P-selectin Leader peptide
atggccaactgccaaatagccatcttgtaccagagattccagagagtggtctttggaatttcccaactcctttgcttcagtgccctgatctctgaactaacaaaccagaaagaagtggcagca

SEQ ID NO: 38 - DNA, 180 bp, Homo sapiens nucleotide sequence encoding the P-selectin Transmembrane cytoplasmic domain
ctgacttactttggtggagcggtggcttctacaataggtctgataatgggtgggacgctcctggctttgctaagaaagcgtttcagacaaaaagatgatgggaaatgccccttgaatcctcacagccacctaggaacatatggagtttttacaaacgctgcatttgacccgagtccttaa

SEQ ID NO: 39 - DNA, 51 bp, Homo sapiens nucleotide sequence encoding the tyrosinase Leader peptide
atgctcctggctgttttgtactgcctgctgtggagtttccagacctccgct

SEQ ID NO: 40 - DNA, 93 bp, Homo sapiens nucleotide sequence encoding the tyrosinase Transmembrane cytoplasmic domain
tgtcgtcacaagagaaagcagcttcctgaagaaaagcagccactcctcatggagaaagaggattaccacagcttgtatcagagccatttataa

SEQ ID NO: 41 - DNA, 72 bp, Homo sapiens nucleotide sequence encoding the GLUT4 Leader peptide
atgccgtcgggcttccaacagataggctccgaagatggggaaccccctcagcagcgagtgactgggaccctg

SEQ ID NO: 42 - DNA, 129 bp, Homo sapiens nucleotide sequence encoding the GLUT4 Transmembrane Cytoplasmic domain
gtacctgaaactcgaggccggacgtttgaccagatctcagctgccttccaccggacaccctctcttttagagcaggaggtgaaacccagcacagaacttgagtatttagggccagatgagaacgactga

SEQ ID NO: 43 - DNA, 63 bp, Rattus norvegicus nucleotide sequence encoding the endotubin Leader peptide
atgtgcctgcctagctgcctcctctcaatctgggtcctatttatggctgcacagtctctaggc

SEQ ID NO: 44 - DNA, 201 bp, Rattus norvegicus nucleotide sequence encoding the endotubin Transmembrane cytoplasmic domain
gcagcacccgtgtctgtgccggttgcagtcggaggagccctcctcctcttcctgttgctcctgggccttggaggttggcactggctgcagaagcagcacctcccctgccaaagtacagatgcagcagcctctggctttgacaatatcctcttcaatgcggatcaagttaccctcccagaatcaatcaccagtaacccatag

SEQ ID NO: 45 - DNA, 69 bp, Mus musculus nucleotide sequence encoding the LAMP-1 leader sequence
atggccgcccccggcgcccggaggcccctgctcctgctgctgctggcaggccttgcacatggcgctagc

SEQ ID NO: 46 - DNA, 108 bp, Mus musculus nucleotide sequence encoding the LAMP-1 transmembrane cytoplasmic domain
atgttgatccccattgctgtgggcggtgccctggcagggctggtcctcatcgtcctcatcgcctacctcattggcaggaagaggagtcacgccggctatcagaccatc

SEQ ID NO: 47 - DNA, 105 bp, Homo sapiens nucleotide sequence encoding the tissue plasminogen activator leader sequence
atggatgcaatgaagagagggctctgctgtgtgctgctgctgtgtggagcagtcttcgtttcgcccagccaggttggtgtgcaggacccctgtgtcccgcccctc

SEQ ID NO: 48 - protein, 35 aa, Homo sapiens tissue plasminogen activator leader peptide
MDAMKRGLCCVLLLCGAVFVSPSQVGVQDPCVPPL

SEQ ID NO: 49 - protein, 17 aa, Artificial sequence
  Blo t 5 H-2d-restricted T cell epitope peptide
AELQEKIIRELDVVCAM

SEQ ID NO: 50 - protein, 117 aa, Artificial sequence
  Blo t 5 peptide
QEHKPKKDDFRNEFDHLLIEQANHAIEKGEHQLLYLQHQLDELNENKSKELQEKIIRELDVVCAMIEGAQGALERELKRTDLNILERFNYEEAQTLSKILLKDLKETEQKVKDIQTQ

SEQ ID NO: 51 - protein, 222 aa, Artificial sequence
  Der p 1 peptide
TNACSINGNAPAEADLRQMRTVTPIRMQGGCGSCWAFSGVAATESAYLAYRNQSLDLAEQELVDCASQHGCHGDTIPRGIEYIQHNGVVQESYYRYVAREQSCRRPNAQRFGISNYCQIYPPNVNKIREALAQTHSAIAVIIGIKDLDAFRHYDGRTIIQRDNGYQPNYHAVNIVGYSNAQGVDYWIVRNSWDTNWGDNGYGYFAANIDLMMIEEYPYVVIL

SEQ ID NO: 52 - protein, 37 aa, Mus musculus LAMP-1 transmembrane & cytoplasmic domain
NMLIPIAVGGALAGLVLIVLIAYLIGRKRSHAGYETI

(19) United States
(12) Patent Application Publication
(10) Pub. No.: US 2006/0251667 A1
(43) Pub. Date: Nov. 9, 2006

(54) Recombinant nucleic acid useful for inducing protective immune response against allergens

(76) Inventors: Kaw Yan Chua, Kent Vale (SG); Lip Nyin Liew, Sabah (MY)

Correspondence Address: KING & SPALDING LLP, 1180 PEACHTREE STREET, ATLANTA, GA, 30309, US

(21) Appl. No.: 10/526,120

(22) PCT Filed: Aug. 29, 2003
(86) PCT No.: PCT/SG03/00205

§ 371 Date: Jul. 14, 2005

Related U.S. Application Data
(60) Provisional application No. 60/406,659, filed on Aug. 29, 2002.

(51) Int. Cl. A61K 48/00; C07H 21/04; C12N 15/09; A61K 39/00; C07K 14/47

(52) U.S. Cl. 424/185.1; 514/44; 530/350; 435/69.3; 435/320.1; 435/325; 536/23.2

(57) ABSTRACT

The invention provides a recombinant nucleic acid useful for inducing a protective immune response against an allergen. The recombinant nucleic acid encodes an allergen and a signal peptide that mediates the translocation of the allergen to endoplasmic reticulum and preferably also encodes a second signal peptide that targets the gene to an endosome or a lysosome. The recombinant nucleic acid, when administered to a subject induces a Th 1 type immunity and inhibits IgE production and therefore may be used to prevent and treat an allergic reaction. In various aspects therefore, the invention provides a vaccine and immunogenic composition comprising the recombinant nucleic acid.

### REFERENCE TO RELATED APPLICATIONS

This application claims the benefit of U.S. Provisional Application No, 60/406,659, filed Aug. 29, 2002, He content of which is herein incorporated by reference.

### FIELD OF THE INVENTION

The present invention relates to recombinant nucleic acid useful for inducing protective immune response against allergens and to vaccines comprising the nucleic acid.

### BACKGROUND OF THE INVENTION

A dramatic increase in the prevalence of allergic diseases worldwide in recent years, particularly in developing countries such as the US, Western Europe, Australia, Japan and Singapore, has highlighted the need for new therapeutic and preventive medical reagents and strategies aimed at suppressing or redirecting the immune response induced upon exposure of an atopic individual to an allergen (1-8).

Briefly, activation of an immune response requires the activation of T cells, either cytotoxic T (killer) cells, or T helper cells. Cytotoxic T cells (commonly referred to as CD8+ cells) are responsible for cellular-based immunity. These cells are stimulated by the presentation of antigen epitopes in complex with MHC class I molecules at the surface of an antigen presenting cell. Antigen-activated cytotoxic T cells then induce cytolysis of infected cells presenting the specific antigen epitope. Antigens that enter the MHC class I presentation pathway are usually derived from pathogens that multiply within the cytoplasm of a host cell, such as a virus.

T helper cells (commonly referred to as CD4+ cells) are involved in humoral immunity. T helper cells are activated by the presentation of antigen epitopes in complex with MHC class II molecules, and activated T helper cells produce cytokines that stimulate production of antigen-specific antibodies. Antigens derived from extracellular pathogens, such as bacteria, or that are synthesized within macrophage, typically enter the MHC class II presentation. MHC class II-associated invariant chain, Ii, which escorts newly, synthesized MHC II molecules from endoplasmic reticulum to the endosomal pathway, and signals associated with lysosomal associated membrane protein, LAMP-I, have been used to target antigens to the endosomal system to enhance MHC class II presentation (17 and 18).

Effector T helper cells can be classified as two subpopulations, the Th1 subset, which secretes IFN-?, and the Th2 subset, which secretes IL-4, IL-5 and IL-13. Antigens derived from within macrophage vesicles generally stimulate the Th1 subset, which then induce production of certain IgG-types of antibodies. Extracellular antigens tend to stimulate the production of Th2 cells, which induce B cells to produce IgM, and may subsequently stimulate the production of different isotopes including IgE, as well as inducing certain classes of IgG antibodies.

Allergen-specific IgE is associated with type I hypersensitivity reaction in allergen-induced diseases. The symptoms associated with type I hypersensitivity reaction include asthma, rhinitis, conjunctivitis and atopic dermatitis. It has been reported that CD8+ suppressor T cells may play a regulating role in IgE production.

The use of DNA as a new prophylactic and therapeutic drug against allergen-induced allergic diseases is an extremely attractive approach. To date, DNA treatment of allergic diseases involves the use of various DNA preparations including gene-based vaccines; protein allergens mixed with immunostimulatory oligodeoxynucleotide (ISS-ODN); allergen-ISS-ODN conjugates (AIC); and immunomodulation using ISS-ODN alone. However, the use of ISS-ODN-based vaccines raises a potential risk of inducing autoimmune reactions in the host (9 & 10). In contrast, the risk of autoimmunity induced by gene-based vaccines appears to be very low (11).

Typically, gene-based vaccines are plasmids that encode a gene for the allergen of interest under control of a strong, broad-specificity eukaryotic promoter, for example the cytomegalovirus (“CMV”) promoter. The DNA is taken up by a wide variety of cells, including antigen presenting cells, which express the allergen, and process it for presentation by way of MHC molecules. Allergens may be derived from a vast variety of sources including dust mites, fungi, pollens, pets, foods, fruits, etc.

Previously, it has been demonstrated that intramuscular injection of laboratory rodents with plasmid encoding an allergen gene results in the induction of Th1 predominant, allergen-specific humoral immunity and cellular immunity (12 & 13). The specific immune response generated by the administration of the allergen gene has been shown to be capable of down-regulating the production of allergen-specific IgE and suppressing the airway hyper-responsiveness in allergen-sensitized animals (12-& 13). However, this approach may not be applicable to all allergens, since some allergens may stimulate a weak IgG₂ₐ-based immune response, or a heightened IgE-based immune response (14-16). Expression of such allergens in vivo could hamper the application of allergen gene immunization in prophylactic and therapeutic treatment against allergen-induced diseases.

Kwon et al. (The effect of vaccination with DNA encoding murine T-cell epitopes on the Der p 1 and 2 induced immunoglobulin E sythesis. S. S. Kwon, N. Kim and T. J. Yoo. 2001, Allergy 56:741-748.) reported that immunization with genes encoding T cell epitopes activates CD8+ cells and inhibits allergen induced IgE synthesis (see also U.S. Pat. Nos. 5,958,891 and 6,251,663) and it has been suggested that activation of CD8+ T cells might confer protection against a subsequent allergic challenge.

Apart from pet Felis domesticus and cockroach, the house dust mite species Dermatophagoides pteronyssinus, (D.p.) Dermatophagoides farinae (D.f.) and Blomia tropicalis (B.t.) are the main triggering factors of indoor allergen-induced diseases. Blomia tropicalis is geographically localized in tropical and subtropical regions whereas Dermatophagoides pteronyssinus is well adapted to temperate, tropical and subtropical areas and Dermatophagoides farinae is more prevalent in cold temperate regions (1&2). The major house dust allergens identified in these species are Der p 1, Der p 2, Der f1, Der f2 and Blo t 5, which are implicated in IgE reactivity in greater than 60% of patients that test positive in mite extract skin prick tests (1-3,6-8). The human IgE specific for major D. p. and D. f. allergens are highly cross-reactive, whereas there is only a small degree of IgE cross-reactivity between B. t. and D. p. allergens. The development of safe and effective vaccines that prevent or treat IgE reaction in allergic disease, and hence type I hypersensitivity reaction, remains an important objective.

### SUMMARY OF THE INVENTION

The invention provides a recombinant nucleic acid comprising a gene encoding a first signal peptide operably linked to a gene encoding an allergen wherein the first signal peptide mediates the translocation of the allergen into the endoplasmic reticulum. In one embodiment, the nucleic acid further comprises an operably linked gene encoding a second signal that targets the allergen, when expressed in the cell to an endosome or lysosome.

The recombinant nucleic acid can be used to induce an immunoprotective response against an allergen and the invention in one aspect provides a vaccine comprising a recombinant nucleic acid according to the present invention. The invention also provides a composition comprising a recombinant nucleic acid or a vaccine according to the invention and a pharmaceutically acceptable carrier or diluent.

The invention in other aspects provides methods of i) immunizing a subject against an allergen; ii) inducing a Th 1 type immune response; iii) inhibiting allergen specific Ig E production; iv) preventing or treating an allergic reaction to an allergen comprising administering a recombinant nucleic acid, a vaccine or composition according to the invention. The invention in other aspects provides use of a recombinant nucleic acid, a vaccine or a composition according to the invention to i) immunize a subject against an allergen; ii) induce a Th 1 type immune response; iii) inhibit allergen specific Ig E production; iv) prevent or treat an allergic reaction to an allergen and use for the manufacture of a medicament to i) immunize a subject against an allergen; ii) induce a Th 1 type immune response; iii) inhibit allergen specific Ig E production; iv) prevent or treat an allergic reaction to an allergen. The subject may be a mammal and in one embodiment, the subject is a human.

The invention in another aspect provides a novel method of immunizing a subject against an allergen comprising administering to the subject multiple doses of a nucleic acid comprising an expressible allergen gene in a first phase over a period of time sufficient to induce a long term memory in the subject and in a second phase, administering the allergen. In one embodiment the nucleic acid is administered in the first phase over a period of about a year. In different embodiments, the allergen may be administered in combination with an adjuvant and multiple doses of the allergen may be administered.

### BRIEF DESCRIPTION OF THE FIGURES

FIG. 1 shows a typical Th2 type immune responses in BALB/cJ mice immunized with alum-absorbed recombinant Blo t 5 protein, as measured by the amount of Th2- and Th1-specific cytokines produced, the level of allergen-specific IgE production, and the airway hyperreactivity response.

FIG. 2 shows induction of specific Th1 type humoral responses in BALB/cJ mice by immunized with DNA vaccine encoding the full-length Blo t 5 gene.

FIG. 3 shows induction of specific Th1 humoral and cellular immune responses in BALB/cJ mice by intramuscular injection with a DNA vaccine encoding a chimeric protein comprising the Mus musculus LAMP-1 signal sequence, the Blo t 5 gene fragment encoding 1-2^{d}-restricted Th epitope and the Mus musculus LAMP-1 transmembrane and cytoplasmic domain, and subsequent boosting with alum-absorbed Blo t 5 protein.

FIG. 4 shows induction of specific Th1 type humoral response in BALB/cJ mice via intradermal injection with a DNA vaccine encoding a chimeric protein comprising the Mus musculus LAMP-1 signal sequence, the Blo t 5 gene fragment encoding H-2^{d}-restricted Th epitope and the Mus musculus LAMP-1 transmembrane and cytoplasmic domain, and subsequent boosting with alum-absorbed Blo t 5 protein.

FIG. 5 shows induction of specific Th1 type humoral responses in BALB/cJ mice via intramuscular injection with a DNA vaccine encoding a chimeric protein comprising the Mus musculus LAMP-1 signal sequence, the Blo t 5 gene fragment encoding H-2^{d}-restricted Th epitope and the Mus musculus LAMP-1 transmembrane and cytoplasmic domain, followed by boosting with alum-absorbed Blo t 5 allergen protein and subsequent aerosol administration of Blo t 5 allergen protein.

FIG. 6 shows induction of long-term Blo t 5-specific immunity memory in BALB/cl mice intramusculariy injected with a DNA vaccine encoding a chimeric protein comprising the Mus musculus LAMP-1 signal sequence, the Blo t 5 gene fragment encoding H-2^{d}-restricted Th epitope and the Mus musculus LAMP-1 transmembrane and cytoplasmic domain, and then boosted with alum-absorbed Blo t 5 allergen protein after a prolonged interval.

FIG. 7 shows induction of long-term Blo t 5-specific immunity memory in BALB/cJ mice intramuscularly injected with a DNA vaccine encoding a chimeric protein comprising the Mus musculus LAMP-1 signal sequence, the Blo t 5 gene fragment encoding H-2^{d}-restricted Th epitope and the Mus musculus LAMP-1 transmembrane and cytoplasmic domain, and then boosted with alum-absorbed Blo t 5 allergen protein. The DNA vaccine priming was given in three doses over an extended period of time before boosting was performed.

FIG. 8 shows induction of specific Th1 humoral immune responses in BALB/cJ mice by intramuscular injection with a DNA vaccine encoding a chimeric protein comprising the Mus musculus LAMP-1 signal sequence, the Blo t 5 gene and with or without the Mus musculus LAMP-1 transmembrane and cytoplasmic domain, and subsequent boosting with alum-absorbed Blo t 5 protein,

FIG. 9 shows the induction of Der p 1-specific Th1 type immunity in BALB/cJ mice by intramuscular injection with a DNA vaccine encoding a chimeric protein comprising the Mus musculus LAMP-1 signal sequence, the Der p 1 gene and the Mus musculus LAMP-1 transmembrane and cytoplasmic domain, and subsequent boosting with alum-absorbed Der p 1 protein.

FIG. 10 shows the suppression of Der p 1-specific Th2 cytokine production and the inhibition of airway hyperreactivity to Der p 1 in BALB/cJ mice by gene immunization. Immunization was done by intramuscular injection with a DNA vaccine encoding a chimeric protein comprising the Mus musculus LAMP-1 signal sequence, the Der p 1 gene and the Mus musculus LAMP-1 transmembrane and cytoplasmic domain, and subsequent boosting with alum-absorbed Der p 1 protein.

FIG. 11 shows the production of Th1 specific antibodies raised against Der p 1 in BALB/cJ mice immunized intramuscularly with a DNA vaccine encoding a chimeric protein comprising the Homo sapiens tissue plasminogen activator signal sequence, the Der p 1 gene and and the Mus musculus LAMP-1 transmembrane and cytoplasmic domain, and subsequent boosting with alum-absorbed Der p 1 protein.

FIG. 12 shows the production of Th1 specific antibodies raised against Der p 1 in BALB/cJ mice immunized orally with chitosan-DNA nanoparticles. The nanoparticles contained a DNA vaccine encoding a chimeric protein comprising the Homo sapiens tissue plasminogen activator signal sequence, the Der p 1 gene and and the Mus musculus LAMP-1 transmembrane and cytoplasmic domain. Priming was followed by subsequent boosting with alum-absorbed Der p 1 protein.

### DETAILED DESCRIPTION OF THE INVENTION

The invention provides a recombinant nucleic acid useful in inducing a protective immune response against an allergen. The term allergen as used in this context refers to any antigen that can elicit an allergic reaction predominantly mediated by IgE and Th2 cytokines. Such allergic reactions are also known in the art as type I hypersensitivity reactions. The term allergic reaction or allergic reactions are used broadly to refer to such reactions and to diseases or symptoms associated with such reactions including allergic rhinitis, allergic asthma, anaphylaxis, wheal and flare reaction, eczema, urticaria and dermatitis.

Allergens are usually environmental or food derived proteins, and most are relatively small, highly soluble proteins that are carried on desiccated particles such as pollen grains or mite feces. On contact with the mucosa of the airways, for example the soluble allergen elutes from the particle and diffuse into the mucosa.

The terms protein and peptide as used herein are intended to refer to any chain of amino acids regardless of length or post-translational modification (eg glycosylation or phosphorylation) and the terms protein and peptide, as understood by those skilled in the art, are distinguished only by the fact that the term peptide generally refers to relatively short amino acid sequence.

The recombinant nucleic acid may be DNA or RNA. In one embodiment the recombinant nucleic acid is DNA comprising a gene encoding a first signal peptide operably linked to a gene encoding an allergen wherein the first signal peptide mediates the translocation of the allergen once expressed in the cell, into the endoplasmic reticulum. The gene encoding a first signal peptide may be any sequence that encodes an amino acid sequence that acts as a signal for protein folding machinery within the cell to direct the allergen to which the amino acid sequence is linked, to the endoplasmic reticulum. For example and without limitation, the first signal peptide may be the N-terminal signal sequence from the gene for LAMP-1 human tissue plasminogen activator (see for example SEQ ID NO: 6), lysosomal membrane protein LIMP-II (see for example SEQ ID NOS: 8, 10, 12, 28, 30, 32), (CD4⁺ T Cells Induced by a DNA Vaccine: Immunological Consequences of Epitope-Specific Lysosomal Targeting. Fernando Rodriguez, Stephanie Harkins, Jeffrey M. Redwine, Jose M. De Pereda, and J. Lindsay Whitton, JOURNAL OF VIROLOGY, Vol, 75(21): 10421-10430.2001; The Residues Leu(Ile)⁴⁷⁵-Ile(Leu, Vat, Ala)⁴⁷⁶, Contained in the Extended Carboxyl Cytoplasmic Tail, Are Critical for Targeting of the Resident Lysosomal Membrane Protein LIMP II to Lysosomes. Ignacio V. Sandoval Juan J. Arredondo S, Jose Alcalde, Alfonso Gonzalez Noriegall, Joel Vandekerckhove, Maria A. Jimenezll, and Manuel Rico. The Journal of Biochemistry, Vol. 269(9): 6622-6631, 1994; Targeting of Lysosomal Integral Membrane Protein LIMP II The Tyrosine-Lacking Carboxyl Cytoplasmic Tail Of LIMP II Is Sufficient For Direct Targeting To Lysosomes. Miguel A. Vega, Fernando Rodriguez S V, Bartolome Segui, Carmela Calesll, Jose Alcalde, and Ignacio V. Sandoval. The Journal Of Biological Chemistry, Vol. 266(25): 16269-16272, 1991; Cloning, Sequencing, and Expression of a cDNA Encoding Rat LIMP 11, a Novel 74-kDa Lysosomal Membrane Protein Related to the Surface Adhesion Protein CD36. Miguel A. Vega, Bartolome Segui-Real, Jose Alcalde Garcia, Carmela Cales, Fernando Rodriguez, Joel Vanderkerckchovev, and Ignacio V. Sandoval. THE JOURNAL OF BIOLOGICAL CHEMISTRY, Vol. 266(25): 16818-16824, 1991), DEC-205 (see for example SEQ ID NOS:14, 16, 34, 36) (The Dendritic Cell Receptor for Endocytosis, DEC-205, Can Recycle and Enhance Antigen Presentation via Major Histocompatibility Complex Class II-positive Lysosomal Compartments Karsten Mahnke, Ming Guo, Sena Lee, Homero Sepulveda, Suzy L. Swain, Michel Nussenzweig, and Ralph M. Steinman. The Journal of Cell Biology, Vol. 151(3): 673-683, 2000; Efficient Targeting of Protein Antigen to the Dendritic Cell Receptor DEC-205 in the Steady State Leads to Antigen Presentation on Major Histocompatibility Complex Class I Products and Peripheral CD8⁺ T Cell Tolerance. Laura Bonifaz, David Bonnyay, Karsten Mahnke, Miguel Rivera, Michel C. Nussenzweig, and Ralph M. Steinman. J. Exp. Med. Vol. 196(12): 1627-1638, 2002; cDNA cloning of human DEC-205, a putative antigen-uptake receptor on dendritic cells. Masato Kato, Teresa K. Neil, Georgina J. Clark Christine M. Morris, Ru diger V. Sorg, Derek N. J. Hart. Immunogenetics, 47: 442-450, 1998), P-selectin (see for example SEQ ID NOS:18, 38) (Lysosomal Targeting of P-selectin Is Mediated by a Novel Sequence within Its Cytoplasmic Tail. Anastasia D. Blagoveshchenskaya, John P. Norcott, and Daniel F. Cutler. THE JOURNAL OF BIOLOGICAL CHEMISTRY, Vol. 273(5): 2729-2737, 1998; A Balance of Opposing Signals within the Cytoplasmic Tail Controls the Lysosomal Targeting of P-selectin. Anastasia D. Blagoveshchenskaya, Eric W. Hewitt, and Daniel F. Cutler. THE JOURNAL OF BIOLOGICAL CHEMISTRY, Vol. 273(43): 27896-27903, 1998; Targeting of P-Selectin to Two Regulated Secretory Organelles in PC12 Cells. John P. Norcott, Roberto Solari, and Daniel F. Cutler. The Journal of Cell Biology, Vol. 134(5): 1229-1240, 1996; Structural and Functional Characterization of Monomeric Soluble P-selectin and Comparison with Membrane P-selectin. Shigeru Ushiyama, Thomas M. LaueTl, Kevin L. Moore, Harold P. Erickson, and Rodger P. McEver. THE JOURNAL OF BIOLOGICAL CHEMISTRY, Vol. 268(20): 15229-15237, 1993; Structure of the Human Gene Encoding Granule Membrane Protein-140, a Member of the Selectin Family of Adhesion Receptors for Leukocytes. Geoffrey I. Johnston, Greg A. Bliss, Peter J. Newman and Rodger P. McEver. THE JOURNAL OF BIOLOGICAL CHEMISTRY, Vol. 265(34): 21381-21385, 1990), tyrosinase (see for example SEQ ID NOS:20, 40) (THE JOURNAL OF BIOLOGICAL CHEMISTRY Vol. 274, No. 18, Issue of April 30, pp. 12780-12789, 1999. A Cytoplasmic Sequence in Human Tyrosinase Defines a Second Class of Di-leucine-based Sorting Signals for Late Endosomal and Lysosomal Delivery. Paul A. Calvo, David W. Frank, Bert M. Bieler, Joanne F. Berson, and Michael S. Marks), the glucose transporter GLUT4 (see for example SEQ ID NOS:22, 42) (The cytosolic C-terminus of the glucose transporter GLUT4 contains an acidic cluster endosomal targeting motif distal to the dileucine signal. Annette M. Shewan, Brad J. Marsh, Derek R. Mmelvin, Sally Martin, Gwyn W. Ggouldã and David E. James. Biochem. J. 350: 99-107, 2000; Cloning and Characterization of the Major Insulin-responsive Glucose Transporter Expressed in Human Skeletal Muscle and Other Insulin-responsive Tissues. Hirofumi Fukumoto S, Toshiaki Kayanol, John B. Busel, Yvonne Edwards, Paul F. Pilcb W, Graeme I. Bell, and Susumu Seino. THE JOURNAL OF BIOLOGICAL CHEMISIRY, Vol. 264(14): 7776-7779, 1989), endotubin (see for example SEQ ID NOS:24, 44) (Cytoplasmic Signals Mediate Apical Early Endosomal Targeting of Endotubin in MDCK Cells. K. E. Gokay, R. S. Young and J. M. Wilson. Trafflic, 2: 487-500, 2001; Targeting of an Apical Endosomal Protein to Endosomes in Madin-Darby Canine Kidney Cells Requires Two Sorting Motifs. K. E. Gokay and J. M. Wilson. Traffic, 1: 354-365, 2000), or Nef protein or a functional equivalent meaning any variation in the sequence that does not affect its function of mediating translocation to endoplasmic reticulum, for example allelic variants, conservative amino acid substitutions and substantially homologous sequences as described in more detail below. In one embodiment, the gene encodes an N-terminal signal sequence of LAMP-1 or a functional equivalent. In another embodiment, the gene encodes an N-terminal signal sequence of human tissue plasminogen activator or a functional equivalent.

LAMP-1 is a membrane protein found in lysosomes and endosomes. LAMP-1 contains an N-terminal signal sequence that localizes LAMP-1 to the endoplasmic reticulum, and a C-terminal transmembrane and cytoplasmic domain that targets LAMP-1 to lysosomes and endosomes. A chimeric LAMP-1/human papillomavirus E7 (HPV-16 E7) antigen construct has been previously shown to generate greater E7-specific immune response than vaccinia containing the wild type HPV-16 E7 gene and it has been suggested that targeting an antigen to the endosomal and lysosomal compartments may enhance MHC class II presentation and vaccine potency (18).

In the case of DNA vaccines for allergens, it has been shown that CD8+ cells could down-regulate the ongoing production of IgE while a lack of CD4+ cells had no effect. Further, more CD8+ cells were detected in the lung of the vaccination group than the control. It has been suggested that DNA vaccination might induce endogenous production of allergic protein and upon presentation in the context of MHC class I molecules, activate CD8+ T cells capable of conferring protection against subsequent allergic challenge (see U.S. Pat. Nos. 5,958,891 and 6,251,663, and Kwan et al). In contrast, there was no suggestion that enhancing MHC class II presentation or processing would be advantageous in inhibiting IgE production.

In the present invention, the inventors have made the surprising discovery that targeting an allergen to MHC class II processing and presentation pathway in the vaccination group can induce a strong Th1 immune response, mediated by IgG₂ₐ, while inhibiting Th2 immune response as mediated by IgE when compared to a control group. The inventors have further found that a signal sequence that mediates the translocation of allergen once expressed in the cell to the endoplasmic reticulum is sufficient to induce a Th 1 immune response. Without being limited to any particular theory, it is believed that once in the endoplasmic reticulum, at least some of the allergen is routed to MHC class II processing and presentation.

Preferably, the recombinant DNA further comprises an operably linked gene encoding a second signal peptide wherein the second signal peptide targets the allergen to an endosome or lysosome. This is believed to further enhance presentation of the allergen in the MHC class TI pathway. The gene encoding the second signal peptide may be any sequence that encodes an amino acid sequence that interacts with the cell machinery to target the allergen to which it is attached to a lysosome or an endosome; For example and without limitation, the second signal peptide may be the C-terminal lysosomal/endosomal targeting sequence from the gene for LAMP-1, human tissue plasminogen activator, LIMP-II (see for example SEQ ID NOS:9, 11, 29, 31), DEC-205 (see for example SEQ ID NOS: 13, 15, 33, 35), P-selectin (see for example SEQ ID NOS: 17, 37), human tyrosinase (see for example SEQ ID NOS: 19, 39), the glucose transporter GLUT4 (see for example SEQ ID NOS: 21, 41), endotubin (see for example SEQ ID NOS: 23, 43) or Nef protein, or a functional equivalent meaning any variation in the sequence that does not affect its function of targeting to an endosome or lysosome, for example allelic variants, conservative amino acid substitutions and substantially homologous sequences as described in more detail below. In one embodiment, the gene encodes the transmembrane and cytoplasmic domain of LAMP-1 or a functional equivalent.

The gene encoding the allergen as that term is used refers to any gene encoding a full length allergen, a T helper cell epitope thereof or an antigenic fragment thereof containing one or more T helper cell epitopes, or a functional equivalent. The allergen includes mite allergens, glutathione S-transferase, pollen, animal dander, house dust and peanut. It is an accepted practice in the field of immunology to use fragments and variants of antigens as vaccines, as all that is required to induce an immune response to a protein is a small (e.g. 8 to 10 amino acid) immunogenic region of the protein. In the case of allergen DNA vaccines, genes that encode T cell epitopes have been shown to be effective vaccines. Useful fragments and T helper cell epitopes may be identified for example using computer-assisted analysis of amino acid sequences as known in the art.

The term “functional equivalent” is used to describe one or more deletion, substitution, modification or addition in the amino acid sequence of the allergen that does not affect the antigenic property of the allergen. In one embodiment, the functional equivalent sequence will differ by one or more conservative amino acid substitutions. Conservative amino acid substitutions are substitutions among amino acids of the same class. These classes include, for example, amino acids having uncharged polar side chains, such as asparagine, glutamine, serine, threonine, and tyrosine; amino acids having basic side chains, such as lysine, arginine, and histidine; amino acids having acidic side chains, such as aspartic acid and glutamic acid; and amino acids having nonpolar side chains, such as glycine, alanine, valine, leucine, isoleucine, proline, phenylalanine, methionine, tryptophan, and cysteine.

The functional equivalent may be naturally occurring, for example, allelic variants or may be designed using known methods for identifying regions of an antigen that are likely to tolerate changes in the amino acid sequence. As an example, allergen from different species are compared and conserved sequences are identified. The more divergent sequences are more likely to tolerate sequence changes. Sequences may also be modified to become more reactive to T- and/or B-cells based on computer-assisted analysis of probable T- or B-cell epitopes. Such functional equivalent of an allergen may be readily identified by immunizing an animal, for example, a mouse with the putative equivalent, challenging the animal with the allergen and determining whether the equivalent confers a protective immune response against the allergen.

In another embodiment, the gene may encode a substantially homologous functional equivalent, meaning that there is a substantial correspondence between the amino acid sequence of the equivalent and the amino acid sequence of the allergen. In specific embodiments, the functional equivalent will be at least about 50%, 75%, 90% and 95% homologous. Homology is measured using sequence analysis software such as Sequence Analysis Software Package of the Genetics Computer Group, University of Wisconsin Biotechnology Center, 1710 University Avenue, Madison, Wis. 53705. Amino acid sequences are aligned to maximize identity. Gaps may be artificially introduced into the sequence to attain proper alignment. Once the optimal alignment has been set up, the degree of homology is established by recording all of the positions in which the amino acids of both sequences are identical, relative to the total number of positions.

In one embodiment, the recombinant DNA comprises a gene encoding house dust mite allergen from species Blomia tropicalis, Dermatophagoides pteronyssinus, or Dermatophagoides farinae. In one embodiment, the allergen is Blo t 1, Blo t 5, Der p 1, Der p 2, Der p3, Der f1, Der f2 or Der f3 or a T helper cell epitope or an antigenic fragment thereof containing one or more T helper cell epitope, or a functional equivalent. Many allergen genes have been cloned and sequenced as described for example in U.S. Pat. Nos. 6,441,157, 6,268,491, 6,214,358, 6,147,201, 6,086,897, 6,077,517, 6,060,057, 5,973,132, 5,876,722, 5,869,288, 5,798,099, 5,773,002, 5,770,202, 5,710,126, 5,556,953, 5,552,142, 5,433,948 and 5,405,758.

Where an amino acid is represented by more than one codon in the genetic code, a given organism may exhibit a particular preference or more common usage of one codon over another. For example, the codons AGO, AGA and CGT all encode arginine. AGG and AGA are used frequently in human coding sequences, while codon CGT is rarely used. Thus, silent mutations within a coding region of DNA made to select a codon preferred for a particular organism, but which result in expression of the same amino acid sequence of an allergen, are included within the scope of the invention and the term “humanized” is used to refer to changes in the gene sequence to select for codons preferred or commonly found in human coding sequences.

The term gene is used in accordance with its usual meaning to mean an operably linked group of nucleic acid sequences. The term recombinant means that something has been recombined such that reference to a recombinant nucleic acid refers to a molecule that is comprised of nucleic acid sequences that are joined together or produced by means of molecular biological techniques. A first nucleic acid sequence is operably linked to a second nucleic acid sequence when the sequences are placed in a functional relationship. For example, a coding sequence is operably linked to a promoter if the promoter activates the transcription of the coding sequence. Similarly, the gene encoding the first signal peptide is operably linked to the gene coding the allergen if upon expression of the recombinant DNA, the signal peptide mediates the translocation of the allergen to the endoplasmic reticulum. Similarly, the gene coding the second signal peptide is operably linked if upon expression of the recombinant DNA the second signal peptide targets the allergen to an endosome or lysosome.

In one embodiment, the gene encoding the first signal peptide is operably linked upstream to the gene encoding the allergen and the gene encoding the second signal peptide is operably linked downstream from the gene encoding the allergen. In specific embodiments, the recombinant DNA comprises one or more sequences of SEQ ID NOS. 2 to 6 and 28 to 48.

The recombinant DNA in one embodiment further comprises a promoter operably linked to drive the expression of the coding sequences. Preferably, the promoter is a strong, broad specificity promoter allowing for high levels of constitutive expression of the coding sequences, for example strong viral promoters such as Rous sarcoma virus (RSV) promoter (Gorman C M, Merlino G T, Willingham M C, Pastan I, Howard B H. The Rous sarcoma virus long terminal repeat is a strong promoter when introduced into a variety of eukaryotic cells by DNA-mediated transfection. Proc Natl Acad Sci USA 1982; 79:6777-6781), SV40 promoter (Ghosh P K, Lebowitz P, Frisque R J, Gluzman Y. Identification of a promoter component involved in positioning the 5′ termini of simian virus 40 early mRNAs. Proc Natl Acad Sci USA 1981; 78:100-104), CMV enhancer or promoter including CMV immediate early (IE) gene enhancer (CMVIE enhancer) (Boshart M, Weber F, Jahn G, Dorsch-Hasler K, Fleckenstein B, Schaffner W. A very strong enhancer is located upstream of an immediate early gene of human cytomegalovirus. Cell 1985; 41:521-530; Niwa H, Yamamura H, Miyazaki J. Efficient selection for high-expression transfectants with a novel eukaryotic vector. Gene 1991; 108: 193-200; see also U.S. Pat. Nos. 5,849,522 and 5,168,062). In one embodiment, the promoter is human CMV promoter.

The DNA sequences of allergens and the signal peptides are known and the recombinant nucleic acid molecule of the present invention may be constructed by standard techniques known to one skilled in the art and described, for example, in Sambrook et al. (2001) in Molecular Cloning: A Laboratory Manual, 3rd Edition, Cold Spring Harbor, Laboratory Press, and other laboratory manuals. In various aspects of the invention, nucleic acid molecules may be chemically synthesized using techniques such as are disclosed, for example, in Itakura et al. U.S. Pat. No. 4,598,049; Caruthers et al. U.S. Pat. No. 4,458,066; and Itakura U.S. Pat. Nos. 4,401,796 and 4,373,071. Such synthetic nucleic acids are by their nature “recombinant” as that term is used herein (being the product of successive steps of combining the constituent parts of the molecule).

In alternative embodiments, isolated nucleic acids may be combined. By isolated, it is meant that the isolated substance has been substantially separated or purified away from other components, such as biological components, with which it would otherwise be associated, for example in vivo, so that the isolated substance may itself be manipulated or processed. The term ‘isolated’ therefore includes substances purified by standard purification methods, as well as substances prepared by recombinant expression in a host, as well as chemically synthesized substances. A promoter is, for example, isolated when it is not immediately contiguous with (i.e., covalently linked to) the coding sequences with which it is normally contiguous in the naturally occurring genome of the organism from which it is derived. A variety of strategies are available for combing or ligating fragments of DNA, and depending on the nature of the termini of the DNA fragments, a suitable strategy will be readily apparent to persons skilled in the art.

Another aspect of the invention provides an expression vector comprising the recombinant nucleic acid molecule of the invention. The vector may be a plasmid or a virus or virus derived. The construction of such a vector by standard techniques will also be well known to one of ordinary skill in the art. The vectors of the present invention may also contain other sequence elements to facilitate vector propagation and selection in host cells for example, coding sequences for selectable markers, and reporter genes, known to persons skilled in the art. In addition, the vectors of the present invention may comprise a sequence of nucleotides for one or more restriction endonuclease recognition sites.

An expression vector of the present invention may be introduced into a host cell, which may include a cell capable of expressing the protein encoded by the expression vector. Accordingly, the invention also provides host cells containing an expression vector of the invention. The term “host cell” refers not only to the particular subject cell but to the progeny or potential progeny of such a cell. Because certain modifications may occur in succeeding generations due to cellular differentiation, mutation or environmental influences, such progeny may not, in fact, be identical to the parent cell, but are still included within the scope of the term as used herein.

Vector DNA can be introduced into cells by conventional transformation or transfection techniques. The terms “transformation” and “transfection” refer to techniques for introducing foreign nucleic acid into a host cell, including calcium phosphate or calcium chloride co-precipitation, DEAE-dextran-mediated transfection, lipofection, electroporation, microinjection and viral-mediated transfection. Suitable methods for transforming or transfecting host cells are well known in the art and can for example be found in Sambrook et al. (Molecular Cloning: A Laboratory Manual, 3rd Edition, Cold Spring Harbor Laboratory press (2001)), and other laboratory manuals.

A cell, tissue, organ, or organism into which has been introduced a foreign nucleic acid, is considered “transformed”, “transfected”, or “transgenic”. A transgenic or transformed cell or organism also includes progeny of the cell or organism and progeny produced from a breeding program employing a transgenic organism as a parent and exhibiting an altered phenotype resulting from the presence of a recombinant nucleic acid construct. A transgenic organism is therefore an organism that has been transformed with a heterologous nucleic acid, or the progeny of such an organism that includes the transgene.

The invention in various aspects provides a transgenic cell and a non-human animal comprising a recombinant nucleic acid molecule according to various embodiments of the invention.

For stable transfection of mammalian cells, it is known that, depending upon the expression vector and transfection technique used, only a small fraction of cells may integrate the foreign DNA into their genome. In order to identify and select these integrants, a gene that encodes a selectable marker (such as resistance to antibiotics) may be introduced into the host cells along with the gene of interest. Preferred selectable markers include those that confer resistance to drugs, such as G418, hygromycin and methotrexate. Nucleic acids encoding a selectable marker may be introduced into a host cell on the same vector as that encoding the peptide compound or may be introduced on a separate vector. Cells stably transfected with the introduced nucleic acid may be identified by drug selection.

The recombinant nucleic acid can be used to induce an immunoprotective response against an allergen, meaning that it induces a predominantly Th 1 type immunity and inhibits IgE production. The invention therefore also provides a vaccine comprising a recombinant nucleic acid according to the invention. A vaccine according to the invention can be used to immunize a subject against particular allergens using the allergen gene or gene fragments to generate immunity. Without being limited by a particular theory, it is believed that by providing for the generation of large quantities of endogenous allergen-specific Th epitopes within a host, the vaccine allows for the enhancement of the priming effect and production of antigen-specific Th1 effector cells. This antigen-specific Th1 microenvironment conferred by the allergen-specific Th1 effector cells is believed to mediate Th1 immune response in the allergen-specific B cells leading to the development of allergen-specific plasma cells upon subsequent allergen challenge.

The invention in other aspects therefore provides methods of i) immunizing a subject against an allergen; ii) inducing a Th 1 type immune response; iii) inhibiting allergen specific Ig E production; iv) preventing or treating an allergic reaction to an allergen comprising administering a recombinant nucleic acid or a vaccine according to various embodiments of the invention. The invention in other aspects provides use of a recombinant nucleic acid or a vaccine according to various embodiments of the invention to i) immunize a subject against an allergen; ii) induce a Th 1 type immune response; iii) inhibit allergen specific Ig E production; iv) prevent or treat an allergic reaction to an allergen and use of a recombinant nucleic acid or a vaccine according to various embodiments of the invention for the manufacture of a medicament to i) immunize a subject against an allergen; ii) induce a Th 1 type immune response; iii) inhibit allergen specific Ig E production; iv) prevent or treat an allergic reaction to an allergen. The subject may be a mammal and in one embodiment, the subject is a human.

In one embodiment, the vaccine is plasmid DNA expression vector. The plasmid may include a eukaryotic origin of replication to ensure maintenance of the vaccine within a host cell. As well, the plasmid may include a prokaryotic origin of replication and a prokaryotic selective gene so as to allow propagation of the plasmid within a prokaryotic host system. Plasmid DNA that has been propagated in a bacterial host is preferable, as the DNA will be unmethylated. Unmethylated CpG dinucleotides in a DNA backbone act as an adjuvant, which may act to stimulate Th1 type immunity.

In another embodiment, the vaccine is a DNA or RNA viral vector. The viral vector may be, for example, adenovirus, adeno-associated virus, herpes virus, vaccinia, or, preferably, an RNA virus such as a retrovirus or an alphavirus. As will be apparent to one skilled in the art, the RNA viral vector upon reverse transcription in infected host cells, provides a recombinant DNA according to the invention.

Live vaccine vectors available in the art include viral vectors such as adenoviruses and poxviruses as well as bacterial vectors, e.g. Shigella, Salmonella, Vibrio cholerae, Lactobacillus, Bacille bilié de Calmette-Guérin (BCG), and Streptococcus.

An example of an adenovirus vector, as well as a method for constructing an adenovirus vector capable of expressing a DNA molecule of the invention, are described in U.S. Pat. No. 4,920,209. Poxvirus vectors include vaccinia and canary pox virus, described in U.S. Pat. No. 4,722,848 and U.S. Pat. No. 5,364,773, respectively. (Also see, e.g., Tartaglia et al., Virology (1992) 188:217) for a description of a vaccinia virus vector and Taylor et al, Vaccine (1995) 13:539 for a reference of a canary pox.) Poxvirus vectors capable of expressing a recombinant nucleic acid of the invention are obtained by homologous recombination as described in Kieny et al., Nature (1984) 312:163 so that the nucleic acid of the invention is inserted in the viral genome under appropriate conditions for expression in mammalian cells. Generally, the dose of vaccine viral vector, for therapeutic or prophylactic use, can be from about 1×10⁴ to about 1×10¹¹, advantageously from about 1×10⁷ to about 1×10¹⁰, preferably of from about 1×10⁷ to about 1×10⁹ plaque-forming units per kilogram. Preferably, viral vectors are administered parenterally, for example, in 3 doses, 4 weeks apart. It is preferable to avoid adding a chemical adjuvant to a composition containing a viral vector of the invention and thereby minimizing the immune response to the viral vector itself.

Non-toxicogenic Vibrio cholerae mutant strains that are useful as a live oral vaccine are known. Mekalanos et al., Nature (1983) 306:551 and U.S. Pat. No. 4,882,278 describe strains which have a substantial amount of the coding sequence of each of the two ctxA alleles deleted so that no functional cholerae toxin is produced. WO 92/11354 describes a strain in which the irgA locus is inactivated by mutation; this mutation can be combined in a single strain with ctxA mutations. WO 94/01533 describes a deletion mutant lacking functional ctxA and attRS1 DNA sequences. These mutant strains are genetically engineered to express heterologous antigens, as described in WO 94/19482. An effective vaccine dose of a Vibrio cholerae strain capable of expressing a polypeptide or polypeptide derivative encoded by a DNA molecule of the invention contains about 1×10⁵ to about 1×10⁹, preferably about 1×10⁶ to about 1×10⁸, viable bacteria in a volume appropriate for the selected route of administration. Preferred routes of administration include all mucosal routes; most preferably, these vectors are administered intranasally or orally.

Attenuated Salmonella typhimurium strains, genetically engineered for recombinant expression of heterologous antigens or not, and their use as oral vaccines are described in Nakayama et al. (Bio/Technology (1988) 6:693) and WO 92/11361. Preferred routes of administration include all mucosal routes; most preferably, these vectors are administered intranasally or orally.

Other bacterial strains used as vaccine vectors in the context of the present invention are described for Shigella flexneri in High et al., EMBO (1992) 11:1991 and Sizemore et al., Science (1995) 270:299; for Streptococcus gordonii in Medaglini et al., Proc. Natl. Acad. Sci. USA (1995) 92:6868; and for Bacille Calmette Guerin in Flynn J. L., Cell. Mol. Biol. (1994) 40 (suppl. I):31, WO 88/06626, WO 90/00594, WO 91/13157, WO 92/01796, and WO 92/21376.

In bacterial vectors, the nucleic acid of the invention is inserted into the bacterial genome or remains in a free state as part of a plasmid.

The present invention also provides a composition comprising a recombinant nucleic acid or a vaccine according to the invention and a pharmaceutically acceptable carrier or diluent. The composition is suitable for methods and uses described above. The composition is therefore an immunogenic composition meaning that it effects an immune response and the invention therefore in one aspect provides an immunogenic composition comprising a recombinant nucleic acid or a vaccine according to various embodiments of the invention and a pharmaceutically acceptable carrier or diluent. The pharmaceutical composition may be adapted for administration, for example, orally, parenterally, nasally, intramuscularly, intravenously, intradermally, intraperitoneally, sublingually, etc.

In one embodiment, the recombinant nucleic acid or vaccine is diluted in a physiologically acceptable solution such as sterile saline or sterile buffered saline, with or without a carrier. When present, the carrier preferably is isotonic, hypotonic, or weakly hypertonic, and has a relatively low ionic strength, such as provided by a sucrose solution, e.g., a solution containing 20% sucrose.

In one embodiment, the recombinant nucleic acid or vaccine may be associated with agents that assist in cellular uptake. Examples of such agents are (i) chemicals that modify cellular permeability, such as bupivacaine (see, e.g., WO 94/16737), (ii) liposomes for encapsulation of the polynucleotide, (iii) cationic lipids or polymers or silica, gold, or tungsten microparticles that associate themselves with the polynucleotides, or (iv) chitosan nanoparticles (see, e.g., J. L. Chew et al. (2003) Vaccine 21: 2720-2729.).

Anionic and neutral liposomes are well-known in the art (see, e.g., Liposomes: A Practical Approach, RPC New Ed, IRL press (1990), for a detailed description of methods for making liposomes) and are useful for delivering a large range of products, including polynucleotides.

Cationic lipids are also known in the art and are commonly used for gene delivery. Such lipids include Lipofectin™ also known as DOTMA (N-[1-(2,3-dioleyloxy)propyl]-N,N,N-trimethylammonium chloride), DOTAP (1,2-bis(oleyloxy)-3-(trimethylammonio)propane), DDAB (dimethyldioctadecylammonium bromide), DOGS (dioctadecylamidologlycyl spermine) and cholesterol derivatives such as DC-Chol (3 beta-(N—(N′,N′-dimethyl aminomethane)-carbamoyl) cholesterol). A description of these cationic lipids can be found in EP 187,702, WO 90/11092, U.S. Pat. No. 5,283,185, WO 91/15501, WO 95/26356, and U.S. Pat. No. 5,527,928. Cationic lipids for gene delivery are preferably used in association with a neutral lipid such as DOPE (dioleyl phosphatidylethanolamine), as described in WO 90/11092 as an example.

Formulation containing cationic liposomes may optionally contain other transfection-facilitating compounds. A number of them are described in WO 93/18759, WO 93/19768, WO 94/25608, and WO 95/02397. They include spermine derivatives useful for facilitating the transport of DNA through the nuclear membrane (see, for example, WO 93/18759) and membrane-permeabilizing compounds such as GALA, Gramicidine S, and cationic bile salts (see, for example, WO 93/19768).

Gold or tungsten microparticles are used for gene delivery, as described in WO 91/00359, WO 93/17706, and Tang et al. Nature (1992) 356:152. The microparticle-coated polynucleotide is injected via intradermal or intraepidermal routes using a needleless injection device (“gene gun”), such as those described in U.S. Pat. No. 4,945,050, U.S. Pat. No. 5,015,580, and WO 94/24263.

Methods of polynucleotide delivery using nanoparticles of the cationic polymer chitosan are known in the art and described, for example, in J. L. Chew et al. (2003) Vaccine 21 2720-2729. Chitosan is a deacylated form of chitin, and may have varying degrees of deacylation. A polynucleotide can be vigorously mixed with chitosan to yield chitosan nanoparticles containing the polynucleotide. Such particles can be used to deliver DNA by oral or mucosal routes of administration.

In one embodiment, the compositions include recombinant nucleic acid or vaccine according to the invention in an effective amount. An “effective amount” refers to an amount effective, at dosages and for periods of time necessary, to achieve the desired therapeutic result, such as a reduction in type 1 hypersensitivity reaction and in turn a reduction in allergic disease progression, or the desired prophylactic result, such as preventing or inhibiting the rate of type 1 hypersensitivity reaction or allergic disease onset or progression.

The amount of DNA to be administered to a subject depends, e.g., on the strength of the promoter used in the DNA construct, the immunogenicity of the expressed gene product, the condition of the subject intended for administration (e.g., the weight, age, and general health of the subject), the mode of administration, and the type of formulation. In general, a therapeutically or prophylactically effective amount from about 100 μg to 5000 μg, preferably, about 200 to 2000 μg of the recombinant nucleic acid in the form of a plasmid is administered to human adults. The administration can be achieved in a single dose or repeated at intervals.

The route of administration may be any conventional route used in the field of vaccines and depends on the formulation selected. The recombinant nucleic acid is advantageously administered via the intramuscular, intradermal, sub-cutaneous or oral routes. When delivered orally, the recombinant nucleic acid may be combined with a jelly, or a similar ingestible substance, so as to enhance ease of delivery.

In accordance with another aspect of the invention, the recombinant nucleic acid, a DNA vaccine or a composition according to the invention may be provided in containers or commercial packages or kits that further comprise instructions for uses described including use thereof to prevent or treat an allergic reaction.

Another aspect of this invention provides a novel vaccination regimen. The regimen comprises an initial priming of a subject's immune response with a recombinant nucleic acid of the invention and subsequent boosting with the allergen. The present invention thus provides a method for immunization against an allergen comprising administering to a subject in a first phase a recombinant nucleic acid according to the invention and in a second phase administering the allergen to the subject. The subject may be any mammal, including human subjects.

The regimen for any particular allergen may be optimized by varying parameters such as dose of DNA, dose of allergen, types of adjuvant, immunization time frame and immunization route, without undue experimentation, as will be within the skill of one of ordinary skill in the art.

Multiple doses of recombinant nucleic acid may be administered. The doses may be administered over a given time span. For example, two or more doses may be administered in the first phase in a period of two days up to about one year. The timing of the administration of the doses may be evenly spaced over the time span, or the doses may be given at irregular intervals over the time span. In one embodiment, at least two doses are administered, about 2 weeks apart. In another embodiment, at least three doses are administered, about one week apart. The multiple doses may be administered over a period of time such that long term immune memory is induced in the subject. For example, in one embodiment, multiple doses are administered in the first phase over a period of about a year.

The allergen may be administered in the second phase in one or more doses in combination with an adjuvant. Preferably, the adjuvant is chosen so as to elicit allergen-specific Th type 1 immune response. Such a response may be measured by the production of Th1 specific immunoglobulins and cytokines. In one embodiment, the allergen is administered in combination with alum.

The amount of allergen and adjuvant to be administered can be determined by routine experimentation by a skilled person. In one embodiment, about 100 ng and 1 mg of allergen is administered, preferably about 1 μg to 100 μg. In a further embodiment, the allergen is administered in combination with about 1 mg to 10 mg of adjuvant, preferably about 2 mg to 5 mg of adjuvant. The allergen or allergen plus adjuvant may be administered by methods commonly known in the art. For example, administration may be oral, sub-lingual, intraperitoneal, nasal, intratracheal, intramuscular, sub-cutaneous, intradermal, etc.

The allergen in the second phase may be administered in one or more doses. The second phase may occur immediately following the first phase, or there may be an interval of time between the last administration of nucleic acid in the first phase and the initiation of administration of allergen in the second phase. If multiple doses of allergen are given, the doses may be administered over a given time span by different administration routes. For example, two or more doses may be administered in a period of two days up to about 10 weeks. The timing of the administration of the doses may be evenly spaced over the time spans or the doses may be given at irregular intervals over the time span.

In one embodiment, the method comprises administration of at least one dose of the allergen by aerosol, preferably, the last dose is given by aerosol.

Thus, in one embodiment of the immunization regimen, Th1 type allergen-specific cellular immunity is primed by immunization with a recombinant nucleic acid of the invention, facilitating the generation of large quantity of endogenous allergen-specific Th epitopes in the first phase. The second phase of the immunization regimen includes a boosting course implemented by intraperitoneal or intraperitoneal and aerosol administration of allergen with adjuvant to the subject, leading to the activation of allergen-specific cellular and humoral immunity and further administration of aerosolized allergen, which can provide an additional level of allergen-specific Th1 humoral immunity.

The vaccination regimen of the invention can be used with any DNA vaccine and is not limited to use with the nucleic acid of the invention. Thus, using any suitable vaccine for an allergen against which immunization is required, a vaccination regimen is provided comprising a first phase of priming with a DNA vaccine encoding an allergen, followed by a second phase of boosting with the allergen as described above. For any given allergen, the regimen may be optimized by varying dose of DNA, dose of allergen, types of adjuvant, immunization time frame and immunization route, without undue experimentation, as will be within the skill of one of ordinary skill in the art.

In one embodiment, a method of immunization is provided comprising administering to a subject in a first phase a nucleic acid comprising an expressible allergen gene; in a second phase administering the allergen to the subject, wherein multiple doses of the nucleic acid are administered in the first phase over a period of about a year so as to induce long term immune memory in the subject. In another embodiment, the method comprises administering the allergen by aerosol in the second phase.

A nucleic acid encodes an expressible allergen gene if, upon delivery to the cells of the subject that is to be vaccinated, the gene product of the allergen gene (the allergen) is expressed within the cells of the subject.

All documents referred to herein are fully incorporated by reference.

Although various embodiments of the invention are disclosed herein, many adaptations and modifications may be made within the scope of the invention in accordance with the common general knowledge of those skilled in this art. Such modifications include the substitution of known equivalents for any aspect of the invention in order to achieve the same result in substantially the same way. All technical and scientific terms used herein have the same meaning as commonly understood by one of ordinary skill in the art of this invention, unless defined otherwise.

The word “comprising” is used as an open-ended term, substantially equivalent to the phrase “including, but not limited to”. The following examples are illustrative of various aspects of the invention, and do not limit the broad aspects of the invention as disclosed herein.

### EXAMPLES

Materials and Methodology

Animals and immunization: Six to eight week old female BALB/cJ mice were purchased from the Laboratory Animal Centre, Lorong Chencharu, Sembawang, Singapore. The animals were kept in a conventional animal room in the NUS Animal Holding Unit. For naked DNA immunization, animals were intramuscularly or intradermally injected with 100 μg of plasmid per dose (given in 2 injections, 50 μl per injection) at day 0, day 7 (for Blot5 minigene injection only) and day 14. On day 21 and either day 42 or day 49, the animals were treated intraperitoneally with appropriate doses of alum-absorbed mite allergen (Blo t 5 or Der p 1). For some particular experiments animals were subjected to a further exposure to aerosolized yeast recombinant dust mite allergen (0.5 mg per ml PBS) at day 63, day 66 and day 69. The sera were collected weekly and stored at −20° C. until assay. The titer and the isotype of the dust mite-specific antiserum were determined by ELISA. Inhalation challenge was performed by exposing animals to allergen aerosol (0.05%) generated by an ultrasonic nebulizer (model UltrNEB 99, DeVilbiss Health Care, Sommerset, Pa.) for 20 minutes. Intratracheal administration was performed by innnoculation of 20 μg Der p 1 in 50 μl PBS at back of the tongue.

Plasmid Preparation: Plasmids were prepared and cloned in bacterial strain E. coli. Propagation of E. coli transformants (DH5α strain) and DNA plasmid purification was done according to the user manual of NucleoBond^{R}PC/BAC kit (MACHEREY-NAGEL, Germany). Purified plasmid DNA was dissolved in phosphate-buffered saline (“PBS”: 0.144 g/L KH₂PO4, 9.00 g/L NaCl, 0.795 g/L Na₂HPO₄.7H₂O) to a final DNA concentration of 2 mg/ml in PBS.

For oral administration using chitosan particles, chitosan (Sigma 22742) was dissolved in 25 mM, pH 5.5 acetic acid to a final concentration of 0.02%. Plasmid DNA was dissolved in 45 mM sodium sulphate. An equal volume of both solutions were mixed to yield nanoparticles. The nanoparticles were observed under the ZEISS Axiovert 25 inverted microscope (Carl Zeiss, NY) and sizing of the nanoparticles was performed by photon correlation spectroscopy on the Zetasizer 3000 (Melvern Instruments Ltd., UK) to obtain average nanoparticle size measurements. Zeta potential of the nanoparticles was measured in demineralised water at neutral pH by laser Doppler anemometry using Zetasizer 3000 (Melvern Instruments Ltd., UK). BALB/c mice in groups of 5 were fed with chitosan-DNA nanoparticles embedded in jelly. Mice were placed into separated cages before feeding and water was given ad libitum. Freshly-made nanoparticles were then mixed in orange-flavoured Lady's Choice jelly crystals (CPC International Inc., Hong Kong) that were dissolved in double-distilled H₂O heated to 50° C. at 150% (w/v) (15 g jelly crystals in 10 ml H₂O). The mixture was then poured onto a weighing tray and left to solidify at 4° C. before being fed to mice. Chitosan nanoparticles containing 50 μg of DNA were mixed in jelly and left for each mouse to eat at day 0. After the mouse had completely consumed the jelly, the mouse was fed standard mouse food.

Gene construction: The expression of all chimeric genes is under the control of the CMV promoter.

Plasmid pCI-Bt5 was generated by insertion of the full length Bt5 cDNA (ref. 18. The gene bank access number is U27479) into the EcoRI and XbaI sites of pCI mammalian expression vector (Promega Corporation).

The control vector pCI-LAMPss-T/C was constructed by insertion of the synthetic oligonucleodide composing the Mus musculus LAMP-1 leader sequence and the Mus musculus LAMP-1 sequence encoding the transmembrane and cytoplasmic tail into the Xho I (the corresponding site in the insert is bolded at the 5′ end of sequence below) and Not I (the corresponding site in the insert is bolded at the 3′ end of sequence below) of pCI vector. A unique Nhe I site and a unique Nde I site were designed at the 3′end of sequence encoding the LAMP-1 leader sequence and at the 5′end of encoding sequence for LAMP-1 transmembrane and cytoplasmic tail, respectively (both underlined). The encoding sequence of the pCI-LAMPss-T/C with the cloning sites into which an allergen gene is inserted is shown below [SEQ ID NO:1]. The translated protein sequences for the mouse LAMP-1 leader sequence and the mouse LAMP-1 transmembrane and cytoplasmic domain are also shown [SEQ ID NO:25, SEQ ID NO:26]:

```
                              M A A P G A R R P L L L L L L A G L A H G
5′ ctcgagccaccatggccgcccccggcgcccggaggcccctgctcctgctgctgctggcaggccttgcacatggc
      A S M L I P I A V G G A L A G L V L
      gctagcgaattcccggggatccatatgttgatccccattgctgtgggcggtgccctggcagggctggtcct
      I V L I A Y L I G R K R S H A G Y E T I
      atcgtcctcatcgcctacctcattggcaggaagaggagtcacgccggctatcagaccatctagcggccgc 3′
```

Plasmid pCI-LAMPss-Bt5₅₀₋₆₇-T/C was constructed using synthetic oligonucleotide composing the Blo t 5 gene fragment that encodes for the H-2^{d}-restricted T epitope. The oligonucleotide was inserted into the Nhe I site at the 3′ end of the Mus musculus LAMP-1 leader sequence and the Nde I site at the 5′ end of the Mus musculus LAMP-1 sequence encoding the transmembrane and cytoplasmic tail. The encoding sequence is [SEQ ID NO:2]

```
                      Mouse LAMP-1 signal sequence
      M A A P G A R R P L L L L L L A G L A H G A S
5′ atggccgcccccggcgcccggaggcccctgctcctgctgctgctggcaggccttgcacatggcgctagc 3′
          Blo t 5 H-2^{d}-restricted T cell epitope
      A E L Q E K I I R E L D V V C A M N
5′ gcagaattgcaagcgaaaatcattcgagaacttgatgttgtttgcgccatgaat 3′
              Mouse LAMP-1 transmembrane & cytoplasmic domain
      M L I P I A V G G A L A G L V L I V L I A Y L
5′ atgttgatccccattgctgtgggcggtgccctggcagggctggtcctcatcgtcctcattgcctacctc
      Mouse LAMP-1 transmembrane & cytoplasmic domain
      I G R K R S H A G Y E T I A M B
      attggcaggaagaggagtcacgccggctatcagaccatctag 3′
```

Plasmid pCI-LAMPss-Bt5-T/C was generated by insertion of PCR amplified Blo t 5 cDNA encoding the mature protein into the Nhe I site at the 3′ end of the Mus musculus LAMP-1 leader sequence and the Nde I site at the 5′ end of the Mus musculus LAMP-1 sequence encoding the transmembrane and cytoplasmic tail. The Blo t 5-LAMP encoding sequence is [SEQ ID NO:3]:

```
                                              Mouse LAMP-I signal sequence
      M A A P G A R R P L L L L L L A G L A H G A S
5′ Atggccgcccccggcgcccggaggcccctgctcctgctgctgctggcaggccttgcacatggcgctagc 3′
                        Blo t 5 encoding sequence
      Q E H K P K K D D F R N E F D H L L I E Q A N H
5′ caagagcacaagccaaagaaggatgatttccgaaacgaattcgatcacttgttgatcgaacaggcaaaccat
      A I E K G E H Q L L Y L Q H Q L D E L N E N K S
      gctatcgaaaagggagaacatcaattgctttacttgcaacaccaactcgacgaattgaatgaaaacaagagc
      K E L Q E K I I R E L D V V C A M I E G A Q G A
      aaggaattgcaagagaaaatcattcgagaacttgatgttgtttgcgccatgatcgaaggagcccaaggagct
      L E R E L K R T D L N I L E R F N Y E E A Q T L
      ttggaacgtgaattgaagcgaactgatcttaacattttggaacgattcaactacgaagaggctcaaactctc
      S K I L L K D L K E T E Q K V K D I Q T Q N
      agcaagatcttgcttaaggatttgaaggaaaccgaacaaaaagtgaaggatattcaaacccaaaat 3′
            Mouse LAMP-1 transmembrsne & cytoplasmic domain
      M L I P L A V G G A L A G L V L I V L I A Y L I
5′ atgttgatccccattgctgtgggcggtgccctggcagggctggtcctcatcgtcctcatcgcctacctcatt
      G R K R S H A G Y E T I
      ggcaggaagaggagtcacgccggctatcagaccatctag 3′
```

Plasmid pCI-LAMPss-Bt5 was derived from pCI-LAMPss-Bt5-T/C by replacement of the Eco RI/Not I fragment encoding for a portion of Blo t 5 and the Mus musculus LAMP-1 transmembrane and cytoplasmic domain with the Eco RI/Not I fragment from pCI-Bt5. The encoding sequence is [SEQ ID NO: 4]:

```
                                              Mouse LAMP-1 signal sequence
      M A A P G A R R I P L L L L L L A G L A H G A S
5′ Atggccgcccccggcgcccggaggcccctgctcctgctgctgctggcaggccttgcacatggcgctagc 3′
                        Blo t 5 encoding sequence
      Q E H K P K K D D F R N E F D H L L I E Q A N H
5′ caagagcacaagccaaagaaggatgatttccgaaacgaattcgatcacttgttgatcgaacaggcaaaccat
      A I E K G E H Q L L Y L Q H Q L D E L N E N K S
      gctatcgaaaagggagaacatcaattgctttacttgcaacaccaactcgacgaattgaatgaaaacaagagc
      K E L Q E K I I R E L D V V C A M I E G A Q G A
      aaggaattgcaagagaaaatcattcgagaacttgatgttgtttgcgccatgatcgaaggagcccaaggagct
      L E R E L K R T D L N I L E R F N Y E E A Q T L
      ttggaacgtgaattgaagcgaactgatcttaacattttggaacgattcaactacgaagaggctcaaactctc
      S K I L L K D L K E T E Q K V K D I Q T Q N
      agcaagatcttgcttaaggatttgaaggaaaccgaacaaaaagtgaaggatattcaaacccaaaattaa 3′
```

Plasmid pCI-LAMPss-Derp1-T/C was generated by insertion of PCR-amplified Der p1 fragment encoding for the mature Der p 1 protein (ref. 20. The gene bank access number is U11695) into the Nhe I site at the 3′ end of the LAMP-1 leader sequence and the Nde I site at the 5′ end of the Mus musculus LAMP-1 sequence encoding the transmembrane and cytoplasmic tail. The encoding sequence is [SEQ ID NO: 5]:

```
                    Mouse LAMP-i signal sequence
    M A A P G A R R P L L L L L L A G L A H G A S
5′atggccgcccccggcgcccggaggcccctgctcctgctgctgctggcaggccttgcacatggcgctagc3′
    (+1) mature Der p 1 encoding sequence
    T N A C S I N G N A P A E A D L R Q M R T V T P I
5′actaacgcctgcagtatcaatggaaatgctccagctgaaatcgatttgcgacaaatgcgaactgtcactcccatt
    R M Q G G C G S C W A F S G V A A T E S A Y L A Y
    cgtatgcaaggaggctgtggttcatgttgggctttctctggtgttgccgcaactgaatcagcttatttggcttac
    R N Q S L D L A E Q E L V D C A S Q H G C H G D T
    cgtaatcaatcattggatcttgctgaacaagaattagtcgattgtgcttcccaacacggttgtcatggtgatacc
    I P R G I E Y I Q H N G V V Q E S Y Y R Y V A R E
    attccacgtggtattgaatacatccaacataatggtgtcgtccaagaaagctactatcgatacgttgcacgagaa
    Q S C R R P N A Q R F G I S N Y C Q I Y P P N V N
    caatcatgccgacgaccaaatgcacaacgtttcggtatctcaaactattgccaaatttacccaccaaatgtaaac
    K I R E A L A Q T H S A I A V I I G I K D L D A F
    aaaattcgtgaagctttggctcaaacccacagcgctattgccgtcattattggcatcaaagatttagacgcattc
    R H Y D G R T I I Q R D N G Y Q P N Y H A V N I V
    cgtcattatgatggccgaacaatcattcaacgcgataatggttaccaaccaaactatcacgctgtcaacattgtt
    G Y S N A Q G V D Y W I V R N S W D T N W G D N G
    ggttacagtaacgcacaaggtgtcgattattggatcgtacgaaacagttgggataccaattggggtgataatggt
    Y G Y F A A N I D L M M I E E Y P Y V V I L N(+222)
    tacggttattttgctgccaacatcgatttgatgatgattgaagaatatccatatgttgtcattctcaat3′
        Mouse LAMP-1 transmembrane & cytoplasmic domain
    M L I P I A V G G A L A G L V L I V L I A Y L I G
5′atgttgatccccattgctgtgggcggtgccctggcagggctggtcctcatcgtcctcatcgcctacctcattggc
    R K R S H A G Y E T I
    aggaagaggagtcacgccggctatcagaccatctag 3′
```

Plasmid pVax-htpa-hDp1-LAMP was generated by insertion of PCR-amplified fragments encoding the leader sequence from human tissue plasminogen activator, humanized Der p1 mature protein and the transmembrane and cytoplasmic tail from Mus musculus LAMP-1 into the BamH I and Xba I sites of pVax (Invitrogen) which is a plasmid vector approved by the FDA for human use. The encoding sequence is [SEQ ID NO: 6]:

```
              human tissue plasminogen activator leader sequence
5′ atg gat gca atg aag aga ggg ctc tgc tgt gtg ctg ctg ctg tgt gga gca gtc ttc gtt tcg ccc
agc cag gtt ggt gtg cag gac ccc tgt gtc ccg ccc ctc 3′
              humanized Der p 1. sequence
5′ acc aac gcc tgc agc atc aac ggc aat gcc ccc gct gag att gat ctg cgc cag atg agg acc gtg
act ccc atc cgc atg caa ggc ggc tgc ggg tct tgt tgg gcc ttc tca ggc gtg gcc gcg acc gag tct
gca tac ctc gcg tat cgg aat cag agc ctg gac ctc gct gag cag gag ctc gtt gac tgc gcc tcc caa
cac ggc tgt cat ggg gat acg att ccc aga ggt atc gaa tac atc cag cat aat ggc gtc gtg cag gaa
agc tat tac cga tac gta gct agg gag cag tcc tgc cgc cgt cct aac gcc cag cgc ttc ggc att tcc
aac tat tgc cag atc tac ccc cct aat gtg aac aag atc agg gag gcc ctg gcg cag acg cac agc
gcc atc gct gtc atc atc gga atc aag gat ctg gac gca ttc cgg cac tat gac ggg cgc aca atc atc
cag cgc gac aac gga tac cag cca aac tat cac gcg gtc aac atc gtg ggt tac tcg aac gcc cag
ggg gtg gac tac tgg atc gtg cgg aac agt tgg gac acc aac tgg ggc gac aac ggc tac ggc tac
ttt gcc gcc aac atc gac ctg atg atg atc gaa gag tac ccg tac gtg gtg atc ctg 3′
                Mouse LAMP-1 transmembrane and cytoplasmic domain
5′ ttg atc ccc att gct gtg ggc ggt gcc ctg gca ggg ctg gtc ctc atc gtc ctc att gcc tac ctc att
ggc agg aag agg agt cac gcc ggc tat cag acc atc tag 3′
```

Production of recombinant Blo t 5 allergen and purification of native Der p 1: Two different expression systems, the E. coli based GST Gene Fusion System and the yeast based Pichia Expresssion System were employed to express the recombinant Blot 5 allergen. For the E. coli based expression system, the entire encoding sequence for mature Blo t 5 was subcloned into the vector pGEX-4T (Amersham Phamacia Biotech). In order to obtain recombinant Blo t 5 with post-translation modification properties of the native Blo t 5, the coding sequence for the mature Blo t 5 was subcloned into the pPICZα vector using the EasySelect™ Pichia Expression Kit (Invitrogen™ life technologies). Protein expression and purification were achieved according to the manual provided by the manufacturers. Native Der p 1 was purified from spent mite media using mAb 4Cl by affinity chromatography

In vitro primary or secondary stimulation of spleen cells and purified T cells: Unpurified spleen cells were used for secondary Blo t 5-specific T cell proliferation assay. Nylon wool purified T cells from spleen suspension were used for primary Blo t 5-specific T cell proliferation culture. Briefly, 1.5×10⁵ purified T cells and 4.5×10⁵ mitomycin-treated APCs were co-cultured in 96-well U bottom plate in the presence or absence of 20 μg of GST-Blo t 5 for 3 to 4 days. The culture supernatant was collected at day 2 and day 3 and stored at −80° C. until ELISA assays for mouse INF-γ and mouse IL-4 were performed. For the secondary re-stimulation culture, 2˜3×10⁷ splenocytes were cultured in 6-well plate in the presence of 20 μg of GST-Blo t 5 for 4 days. Ficoll-Plaque-purified T cells were collected and maintained for additional 6 days in the presence of 20 ng per ml of mouse recombinant IL-2 in RP10 medium. 1×10⁵ viable T cells were loaded onto well (96-well U bottom plate) pre-coated with anti-mouse CD3ε antibody and were cultured in the presence or absence of 1 mg per ml of anti-mouse CD28 antibody for additional 24 hours or 48 hours. The 24-h or 48-hour culture supernatant was collected and stored at −80° C. until use in IL-4 ELISA assays.

Immunoglobulin and cytokine ELISA: A Costar high binding 96-well ELISA plate was coated with GST-Blo t 5, native Der p 1, rat anti-mouse IL-4, or rat anti-mouse IFNγ (2-5 μg/ml) overnight at 4° C. After blocking the wells with 10% FCS or 1% BSA, appropriately diluted sera/culture supernatant were added and plates were subjected to overnight incubation at 4° C. Biotin-conjugated mAb, rat anti-mouse IgE, rat anti-mouse IgG₂ₐ, rat anti-mouse IL-4, or rat anti-mouse IFNγ were added, followed by ExtrAvidin-alkaline phosphatase. The signal was developed by p-Nitrophenylphosphate substrate and the optical density was measured at OD405 nm. Mouse IgE, IgG₂ₐ, recombinant mouse IL-4 & IFNγ were used as standards.

Measurement of airway responsiveness: Airway responsiveness was assessed by methacholine-induced airflow obstruction on conscious animals using a whole-body plethysmography (model PLY3211, Buxco Electronics Inc., Troy, New York, USA). Allergen-challenged animals were first exposed to PBS for baseline measurement following by cumulative increased doses of aerosolized methacholine. The measurement index is denoted as Penh according to the equation Penh=(Te/RT−1)×(PEF/PIF) where Penh=enhanced pause, Te=expiratory time, RT=relaxation time, PEF=peak expiratory flow, and PIF=peak inspiratory flow (21).

### Example 1

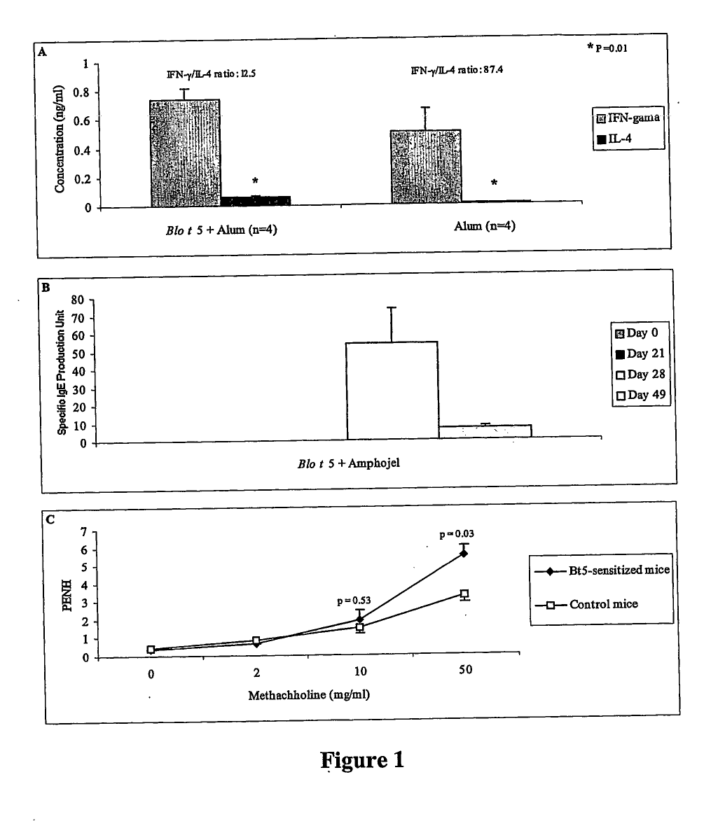

Six to eight week old animals (n=4 per group) were intraperitoneally administered 10 μg and 5 μg of yeast recombinant Blo t 5 in 4 mg of alum (Amphojel^{R}) at day 0 and day 21, respectively. The sera were collected weekly and stored at −20° C. until ELISA assays could be performed. The levels of Blo t 5-specific IgE anti-sera were determined by ELISA. One antibody production unit corresponds to one nanogram of mouse Ig per ml of serum (FIG. 1A). Single spleen cell suspension was prepared at day 21 from mice pre-primed with 10 μg alum-absorbed Blo t 5 or alum alone (day 0). Splenocytes were stimulated with Bt5₅₀₋₆₇ peptide (5 μM for 72 hours. The levels of IFNγ and IL-4 in the culture supernatants were determined by ELISA (FIG. 1B). Six to eight week old animals (n=3 or 4 per group) were intraperitoneally administrated with 10 μg and 5 μg of yeast recombinant Blo t 5 in 2 mg of alum at day 0 and day 21, respectively. The animals were further boosted with Blo t 5 aerosol (0.025%) at day 28, day 31 and day 34. Airway hyperreactivity measurement was tested at day 35 (FIG. 1C).

The results indicate successful establishment of an allergen-induced mouse model having Th2 type immunity characteristics. IL-4 is the key cytokine that regulates the synthesis of IgE. A statistically significant level of IL-4 (P=0.01) was secreted by splenocytes from animals primed with alum-absorbed Blo t 5 upon in vitro stimulation with H-2^{d}-restricted Blo t 5 Th epitopes as compared with the control group (FIG. 1A). Administration of a further booster of alum-absorbed Blo t 5 at day 21 resulted in an immediate surge of Blo t 5-specific IgE level at day 28, followed by a sharp fall in the Blo t 5-specific titer (FIG. 1B). The magnitude of Blo t 5-specific IgE titer in the experimental group was persistently above the background level for more than 6 weeks (data not shown). Animals characterized with Th2 type Blo t 5-specific immunity exhibited a statically significant asthmatic symptom comparing to the control animals after exposure to methacholine aerosol (P=0.03) (FIG. 1C). Thus, this immunization protocol by using alum as a Th2 adjuvant is feasible to induce a long-lasting and significant Blo t 5-specific Th2 type immunity in BALB/cJ animals that characterized with asthmatic symptoms.

### Example 2

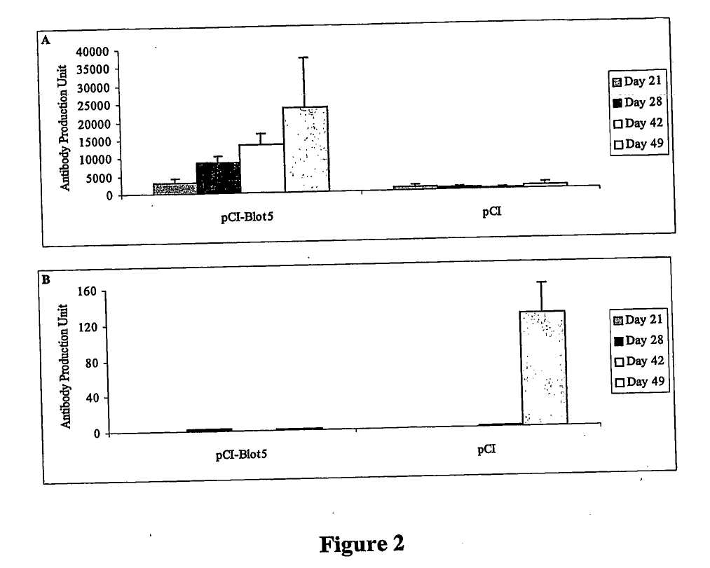

Six to eight weeks old animals (n=4 per group) were intramuscularly injected with 100 μg of pCI-Blot5 and pCI at day 0 and 14. Subsequently the animals were intraperitoneally treated twice with 10 μg (day 21) and 5 μg (day 42) of yeast recombinant Blo t 5 allergen in 4 mg of alum. The sera were collected weekly and stored at −20° C. until assay. The levels of Blo t 5-specific IgG₂ₐ (FIG. 2A) and IgE (FIG. 2B) anti-sera were determined by ELISA. One antibody production unit corresponds to one nanogram of mouse Ig per ml of serum.

Immune responses of animals that received intramuscular naked gene immunization and alum-absorbed Blo t 5 booster are shown in FIG. 2. As shown, Blo t 5 full gene immunization was able to mount a Th1-predominant immune response in animals that received three intramuscular injections of pCI-Blot5, as seen by the appearance of significantly elevated levels of Blo t 5-specific serum IgG₂ₐ as early as day 21 (FIG. 2A). No Blo t 5-specific serum Ig was detected at day 21 in animals injected with the control pCI vector. In contrast to the prominent Th2 type immunity profile elicited in pCI-immunized mice (FIG. 2B), Th1 type immune response was persistently maintained in pCI-Blot5-immunized animals following protein sensitization with yeast recombinant Blo t 5 in alum (FIG. 2A). These results were consistent with the in vitro T cell cytokine profiles, with a greater than three-fold INF-γ/IL-4 ratio for the experimental animals as compared with that of the control animals (data not shown).

### Example 3

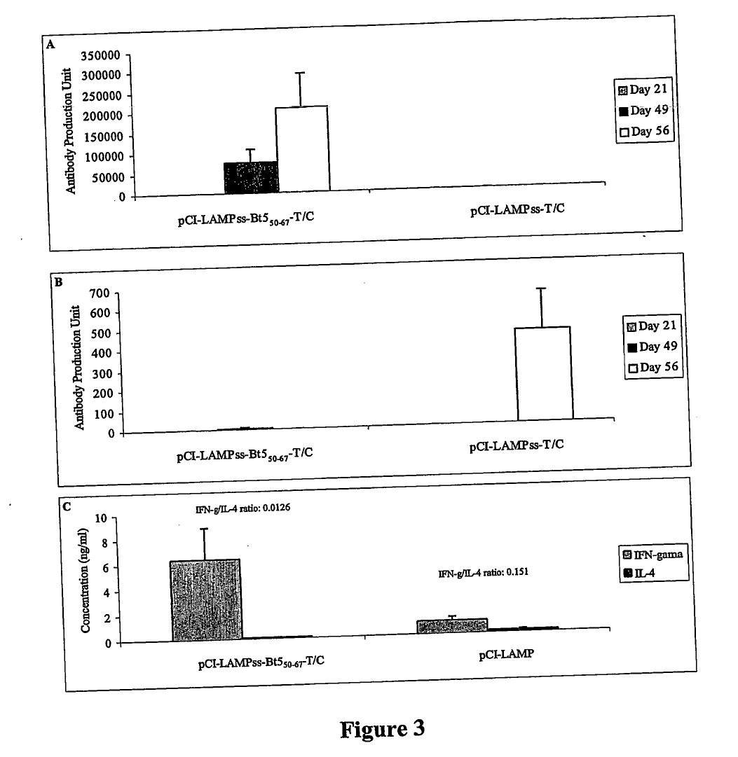

Enhancing DNA vaccine potency can be achieved by (1) targeting the T helper cell epitope to the MHC II pathway, and (2) optimizing the immunization timeframe, immunization route, and appropriate adjuvant, as demonstrated by the results depicted in FIGS. 3 to 7.

Six to eight week old animals (n=4 per group) were intramuscularly injected with 100 μg of pCI-LAMPss-Bt5₅₀₋₆₇-T/C or pCI-LAMPss-T/C at day 0 and 14. Subsequently the animals were treated intraperitoneally twice with 10 μg and 5 μg of yeast recombinant Blo t 5 allergen in 4 mg of alum at day 21 and day 49, respectively. The sera were collected weekly and stored at −20° C. until assay. The levels of Blo t 5-specific IgG₂ₐ (FIG. 3A) and IgE (FIG. 3B) anti-sera were determined by ELISA. One antibody production unit corresponds to one nanogram of mouse Ig per ml of serum. In a second set of experiments, the same immunization protocol was employed except that each individual animal (n=4 per group) was treated intraperitoneally twice with 10 μg and 5 μg of yeast recombinant Blo t 5 allergen in 2 mg of alum at day 21 and day 42, respectively. Splenocytes prepared at day 49 were stimulated with recombinant Blo t 5 (10 μg/ml) for 72 hours. The levels of IFNγ and IL-4 presented in the culture supernatants were determined by ELISA (FIG. 3C).

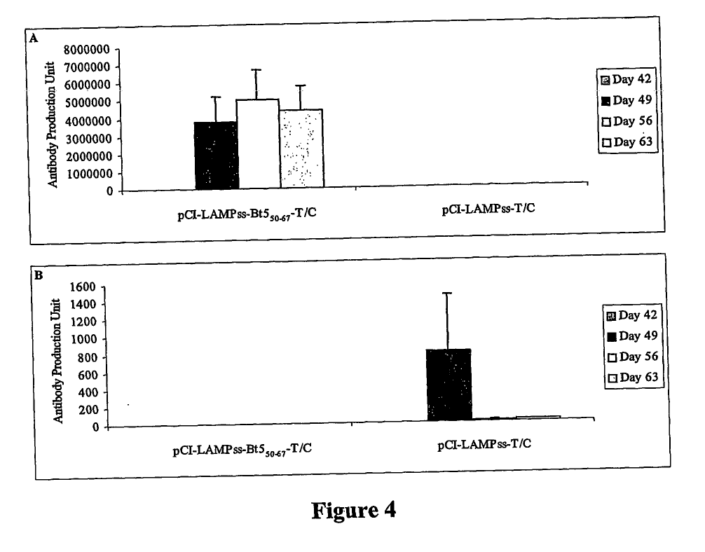

In an additional experiment (FIG. 4), animals were given intradermal injections with 100 μg of pCI-LAMPss-Bt5₅₀₋₆₇-T/C or pCI-LAMPss-T/C at day 0, 7 and 14. All other parameters were as described for the above experiment. The levels of Blo t 5-specific IgG₂ₐ(FIG. 4A) and IgE (FIG. 4B) anti-sera were determined by ELISA.

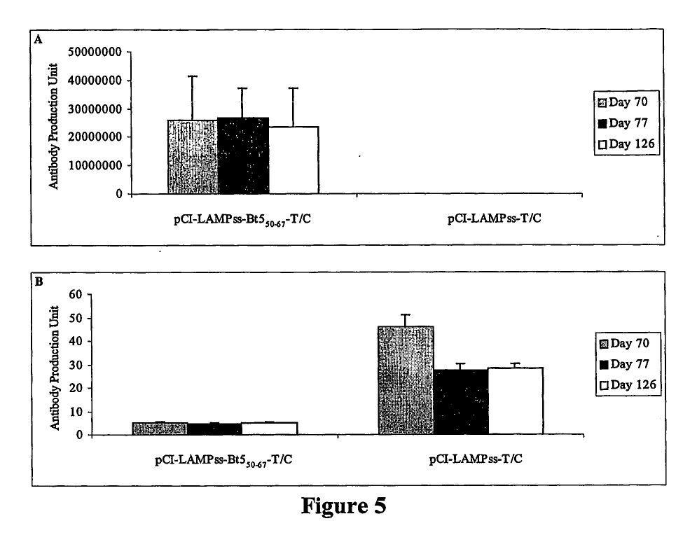

In another set of experiments (FIG. 5), all animals subsequently received additional yeast recombinant Blo t 5 aerosol treatment at day 63, day 66, and day 69. The sera were collected weekly and stored at −20° C. until assay. The levels of Blo t 5-specific IgG₂ₐ(FIG. 5A) and IgE (FIG. 5B) anti-sera were determined by ELISA.

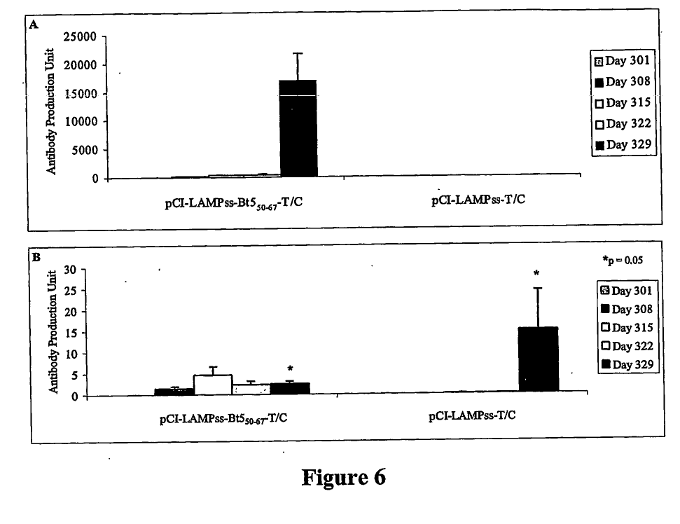

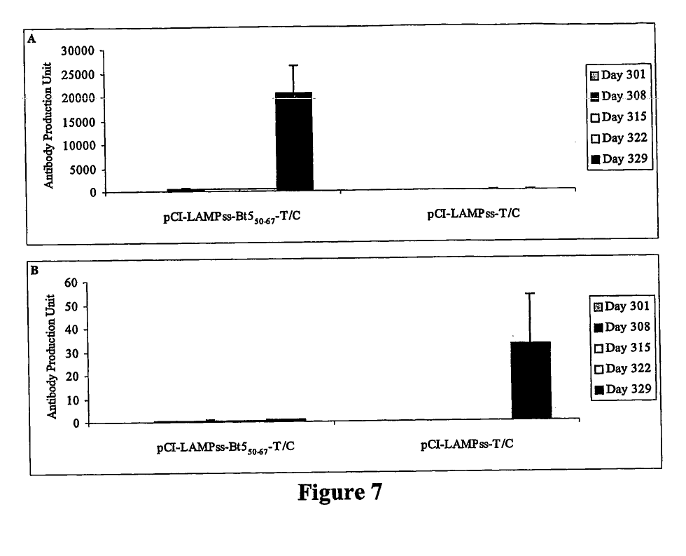

Six to eight week old animals (n=6 per group) were intramuscularly injected with 100 μg of pCI-LAMPss-Bt5₅₀₋₆₇-T/C or pCI-LAMPss-T/C at day 0 and 14 (FIG. 6) or at day 0, day 14 and day 294 (FIG. 7). The animals were intraperitoneally boosted twice with 10 μg and 5 μg of yeast recombinant Blo t 5 allergen in 2 mg of alum at day 301 and day 322, respectively. The sera were collected weekly and stored at −20° C. until assay. The levels of Blo t 5-specific IgG₂ₐ(FIGS. 6A, 7A) and IgE (FIGS. 6B, 7B) anti-sera were determined by ELISA.

FIG. 3 shows specific Th1 humoral immune responses in BALB/cJ mice primed with Blo t 5 minigene and boosted with alum-absorbed Blo t 5. Although alum is considered a Th2-driven adjuvant, a dramatic increase in titer of Blo t 5-specific serum IgG₂ₐ was elicited in animals that received pCI-LAMPss-Bt5₅₀₋₆₇-T/C but not pCI-LAMPss-T/C vector, when followed by two boosters of alum-absorbed Blo t 5 (FIG. 3A). IgG₂ₐ is a typical Th1 type immunoglobulin. In contrast, the control group animals that were immunized with pCI-LAMPss-T/C vector expressed a typical Th2 immunity with significant level of Blo t 5-specific circulating IgE (FIG. 3B). This humoral immunity difference is closely correlated to the Th1/Th2 cytokine profile results obtained from in vitro stimulation of splenocytes with Blo t 5, as indicated by a five-fold difference (0.5995/0.01223) between the IFNγ/IL-4 ratio of the pCI-LAMPss-Bt5₅₀₋₆₇-T/C-immunized group comparing with the control pCI-LAMPss-T/C-immunized group (FIG. 3C). In comparing the results of FIG. 2 and FIG. 3, it can be seen that a more than twenty-fold magnitude of Th1 humoral immunity was elicited in animals immunized with pCI-LAMPss-Bt5₅₀₋₆₇-T/C than in those immunized with pCI-Blot5.

Intradermal DNA immunization is an alternative route that can achieve high levels of protective immunity against allergen-induced diseases. Like intramuscular injection, intradermal injection of pCI-LAMPss-Bt5₅₀₋₆₇-T/C in-vivo is capable of priming substantial Th1-predominant immunity. Upon sensitization with yeast recombinant Blo t 5 in alum a comparable level of Blo t 5-specific serum IgG₂ₐ (FIGS. 3A & 4A) was expressed in animals treated with pCI-LAMPss-Bt5₅₀₋₆₇-T/C but not in animals treated with vector pCI-LAMPss-T/C (FIG. 4B). These results suggest that intradermal DNA immunization could be an alternative route to elicit high quantity of specific Th1 type immunity.

Allergen aerosol inhalation is an effective boosting route to raise enormous protective Th1 immunity in mice. Aerosol inhalation is a natural route to boost the antigen-specific immunity or to induce antigen-specific tolerance in vivo. The feasibility of aerosol inhalation as an antigen-boosting route was investigated by exposing the animals to yeast recombinant Bt5 aerosol. After three consecutive aerosol inhalations of yeast recombinant Blo t 5, animals with significant allergen-specific Th1 immunity (as per FIG. 3A) displayed a remarkable level of allergen-specific serum IgG₂ₐ (more than a 100-fold increment, FIG. 5A) above the basal level of allergen-specific serum IgE. In contrast, control group animals maintained a steady and significant level of allergen-specific serum IgE with basal levels of allergen-specific serum IgG₂ₐ(<200 antibody production units, FIG. 5B). These results suggest that a further boosting effect of the existing Th1 immunity could be achieved by aerosol inhalation of the antigen. Therefore, administration route is a key factor for improving the potency of DNA prime/protein boost regimen.

An ideal vaccine should not only be able to induce a strong and effective but also a long-lasting immunity in the host. FIG. 6 shows the maintenance of long-term immunity memory. During a 10-month resting course study, pCI-LAMPss-Bt5₅₀₋₆₇-T/C-immunized animals were capable of inducing a high level of Blo t 5-specific Th1 immunity (FIG. 6A) following boosting with alum-absorbed Blo t 5, as well as the elicitation of some level of Th2 immunity (FIG. 6B). These results may imply the existing Blo t 5-specific Th1 memory or effector subsets are somewhat quantitatively and/or qualitatively impotent to prevent the polarization of some new Blo t 5-specific Th2 cells in pCI-LAMPss-Bt5₅₀₋₆₇-T/C-immunized animals during the boosting by alum-absorbed Blo t 5.

FIG. 7 shows the immunity boost-up of the long-resting memory by a new immunization protocol. Prior to alum-absorbed Blo t 5 booster, an additional pCI-LAMPss-Bt5₅₀₋₆₇-T/C-immunization was conducted at day 294 (FIG. 7). As expected, a much lower level of Blo t 5 IgE was seen in pCI-LAMPss-Bt5₅₀₋₆₇-T/C-immunized group with a significant reduction at day 329 (=0.05) compared with the result shown in FIG. 6B. Both immunization protocols were able to elicit very similar levels of Blo t 5-specific Th1 immunity (FIGS. 6 & 7). Taken together, the results suggest that expression of a Th dominant Blo t 5 epitope in vivo by intramuscular injection is capable of eliciting a long lasting Blo t 5-specific Th1 dominant immunity. Furthermore, the time frame of DNA-priming followed by protein-boosting could be one of the key parameters for DNA vaccine optimization.

### Example 4

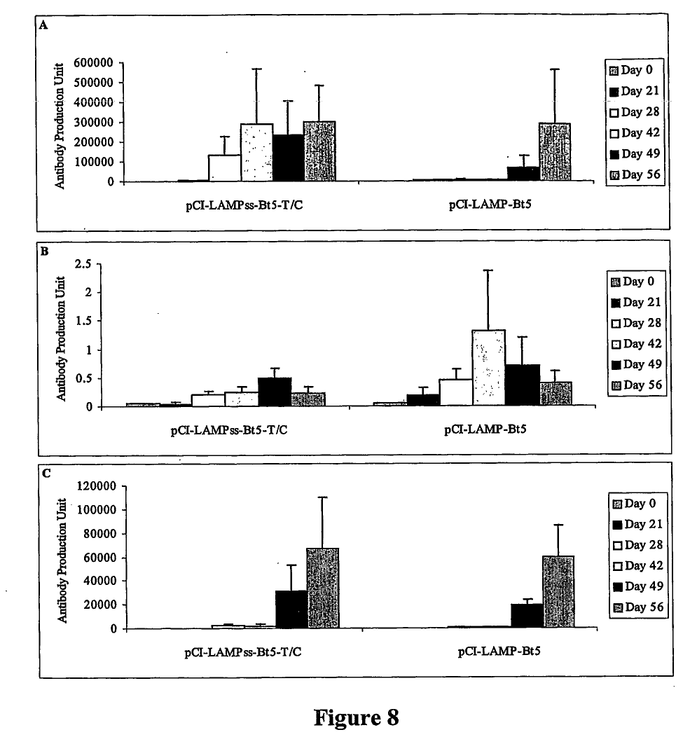

Six to eight week old animals (n=4 per group) were intramuscularly injected with 100 μg of pCI-LAMPss-Bt5-T/C or pCI-LAMPss-Bt5 at day 0, day 7, and day 14. Subsequently the animals were treated intraperitoneally twice with 10 kg and 5 μg of yeast recombinant Blo t 5 allergen in 2 mg of alum at day 21 and day 42, respectively. The sera were collected weekly and stored at −20° C. until assay. The levels of Blo t 5-specific IgG₂ₐ(FIG. 8A), IgE (FIG. 8B), and IgG₁ (FIG. 8C) anti-sera were determined by ELISA. One antibody production unit corresponds to one nanogram of mouse Ig per ml of serum.

FIG. 8 shows specific Th1 humoral immune responses in BALB/cJ mice first primed with lysosome-targeting or lysosome-non-targeting Blo t 5-LAMP chimeric genes and subsequently boosted with alum-absorbed Blo t 5. An earlier protective IgG₂ₐ immune response was elicited in the pCI-LAMPss-Bt5-T/C-immunized animals comparing with that of pCI-LAMPss-Bt5-immunized animals. Results show that a single dose of Blo t 5/alum booster was sufficient to induce comparable level of Blo t 5-specific IgG₂ₐ in pCI-LAMPss-Bt5-T/C-immunized animals, whereas two doses were required in pCI-LAMPss-Bt5-immunized animals.

### Example 5

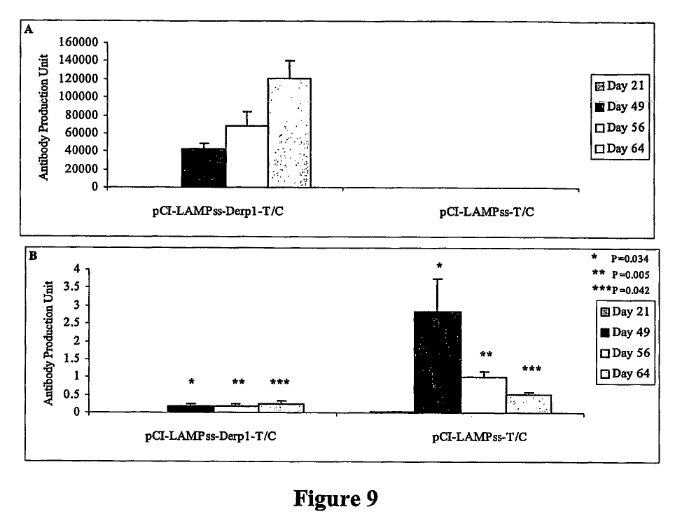

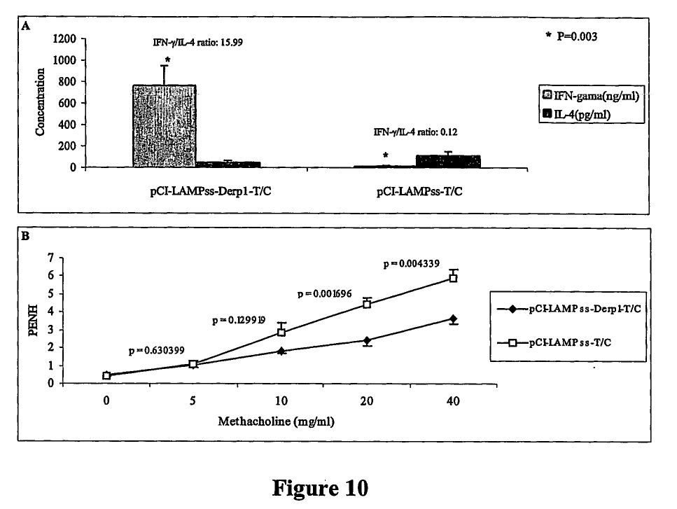

FIGS. 9 and 10 show the effects of suppression of Der p 1-specific IgE production and inhibition of airway hyperresponsiveness to Der p 1 challenging in mice immunized with Derp 1-LAMP chimeric gene.

Six to eight weeks old animals were intramuscularly injected with 100 μg of pCI-LAMPss-Derp1-T/C (n—10) or pCI-LAMPss-T/C (n=6) at day 0 and day 14. The animals were intraperitoneally boosted twice with 1 μg of native Der p 1 in 2 mg of alum at day 21 and day 42. Subsequent intratracheal administration of 20 μg of native Der p 1 was carried out at day 63. The sera were collected weekly and stored at −20° C. until assay. The levels of Der p 1-specific IgG₂ₐ (FIG. 9A) and IgE (FIG. 9B) anti-sera were determined by ELISA. One antibody production unit corresponds to one nanogram of mouse Ig per ml of serum. The animals were subjected to airway hyperreactivity measurement at day 64 (FIG. 10B) and, cytokine reactive profiles of secondary T cells to native Der p 1 (10 μg/ml) were determined by ELISA (FIG. 10A).

FIG. 9 shows the induction of Th1 humoral immunity by animals immunized with Derp1-LAMP chimeric gene. Likes Blo t 5 Th epiopte DNA immunization, high levels of Der p 1-specific IgG₂ₐ was detected in the pCI-LAMPss-Der p 1-T/C-immunized group but not in the control group, following two doses of alum-absorbed Der p 1 booster (FIG. 9A). In contrast, a significant level of Der p 1-specific IgE was expressed in the control group but not in the experimental group (FIG. 9B).

Production of Der p 1-specific Th2 cytokine is suppressed and airway hypersensitivity to Der p 1 is inhibited in mice immunized with Derp1-LAMP chimeric gene. In vitro T-cell proliferation assays indicate that the pCI-LAMPss-Derp1-T/C-immunized group exhibited a typical Th1 profile with high levels of IFNγ and low levels of IL-4, while the pCI-LAMPss-T/C-immunized group displayed a typical Th2 profile with low levels of IFNγ and high levels of IL-4 (FIG. 10A). The pCI-LAMPss-T/C-immunized control group developed statistically significant airway hypersensitivity as compared to the pCI-LAMP-Derp1-T/C experimental group after administration of 20 mg/ml and 40 mg/ml of methacholine (FIG. 10B; P=0.0017 & P=0.0043). Taken together, these results suggest that Derp1-LAMP DNA vaccination is capable of eliciting protective immunity against experimental induced Der p 1 asthma in mice.

### Example 6

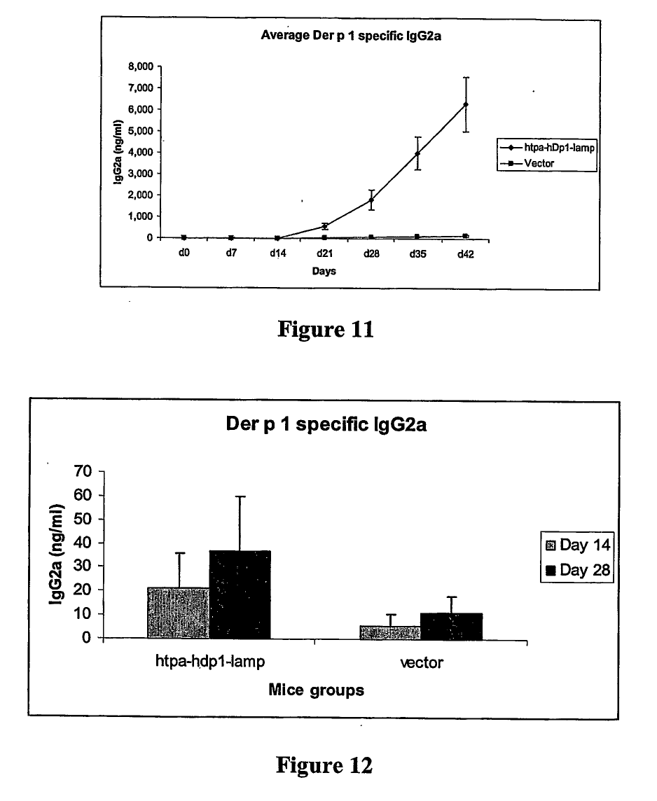

Female BALB/c (n=5) mice were immunized intramuscularly with 50 ug of plasmid DNA (pVax-htpa-hDp1-LAMP) or pVax vector control, with eletroporation at days 0 and 7. Mice were given booster injection with 25 ug Der p1 protein in 2 mg of alum intraperitoneally at day 14. Mice were bled weekly from d0 to d42. Serum IgG₂ₐ specific to Der p1 was measured by ELISA (FIG. 11).

In another set of experiments, BALB/cJ (n=5) mice were fed jelly containing chitosan-DNA with 50 μg of either pVax-htpa-hDp1-LAMP or pVax control vector at day 0. Mice were given booster injection with 25 ug Der p1 protein in 2 mg of alum intraperitoneally at day 14. Mice were bled weekly from d0 to d42. Serum IgG₂ₐ specific to Der p1 was measured by ELISA (FIG. 12).

Mice immunized intramuscularly (FIG. 11) or orally (FIG. 12) produced Der p 1 specific IgG₂ₐ antibodies. As well, these results indicate that oral feeding of chitosan-DNA nanoparticles encoding the Der p 1 gene could raise a Th1 specific immune responses against Der p 1 allergen.

1. A recombinant nucleic acid comprising a gene encoding a first signal peptide operably linked to a gene encoding an allergen wherein the first signal peptide mediates the translocation of the allergen into the endoplasmic reticulum.

2. The nucleic acid of claim 1 which is DNA.

3. The nucleic acid of claim 1 wherein the first signal peptide is the N-terminal signal peptide of LAMP-1, human tissue plasminogen activator, LAMP-II, DEC-205, P-selectin, tyrosinase, GLUT4, endotubin or Nef protein or a functional equivalent thereof.

4. The nucleic acid of claim 1 wherein the first signal peptide is the N-terminal signal peptide of LAMP-1 or human tissue plasminogen activator or a functional equivalent thereof.

5. The nucleic acid of claim 1 further comprising an operably linked gene encoding a second signal peptide wherein the second signal peptide targets the allergen to an endosome or lysosome.

6. The nucleic acid of claim 5 wherein the second signal peptide is the C-terminal lysosome or endosome targeting sequence of LAMP-1, human tissue plasminogen activator, LAMP-II, DEC-205, P-selectin, tyrosinase, GLUT4, endotubin or Nef protein or a functional equivalent thereof.

7. The nucleic acid of claim 6 wherein the second signal peptide is the transmembrane and cytoplasmic domain of LAMP-1.

8. The nucleic acid of claim 1 which encodes the allergen Blo t 5, Blo t 1, Der p 1 or Der p 2, Der p 3, Der f1, Der f2, Der f3, a T helper cell epitope thereof, or a antigenic fragment thereof containing one or more T helper cell epitope or a functional equivalent.

9. The nucleic acid of claim 1 comprising the sequence of SEQ ID NO:2, SEQ ID NO:3, SEQ ID NO:4, SEQ ID NO:5, or SEQ ID NO:6.

10. The nucleic acid of claim 1 which is a plasmid.

11. The nucleic acid of claim 1 further comprising an operably linked promoter.

12. The nucleic acid of claim 11 wherein the promoter is human CMV promoter.

13. The nucleic acid of claim 11 which is an expression vector.

14. A vaccine comprising a recombinant nucleic acid according to any one of claims 1 to 13.

15. A composition comprising a recombinant nucleic acid according to any one of claims 1 to 13 and a pharmaceutically acceptable carrier or diluent.

16. A method for immunization against an allergen comprising administering to a subject in a first phase a recombinant nucleic acid according to any one of claims 1 to 13, and in a second phase administering the allergen to the subject.

17. The method of claims 16 wherein the allergen is administered in combination with an adjuvant.

18. The method of claim 16 wherein the nucleic acid is administered in the first phase over a period of time sufficient to induce long term immune memory in the subject.

19. The method of claim 18 wherein multiple doses of the nucleic acid is administered in the first phase over a period of about a year.

20. The method of claim 16 comprising administering the allergen to the subject intraperitoneally and subsequently by aerosol.

21. The method of claim 16 wherein the nucleic acid is administered orally in the first phase.

22. The method of claim 22 comprising administering chitosan nanoparticles containing the nucleic acid.

23. The method of claim 16 wherein the nucleic acid is administered by intramuscular or intradermal injection.

24. A method for immunization against an allergen comprising administering to a subject a nucleic acid comprising an expressible allergen gene in a first phase over a period of about a year so as to induce long term immune memory in the subject; and administering the allergen to the subject in a second phase.

25. A method for treating or preventing an allergic reaction in a subject comprising administering a recombinant nucleic acid according to any one of claims 1 to 13 to the subject.

26. The method of claim 25 wherein the recombinant nucleic acid is administered orally or by intramuscular or intradermal injection.

27. The method of claim 25 wherein the allergic reaction is asthma or rhinitis.

28. (canceled)

29. (canceled)

30. (canceled)

31. (canceled)

32. (canceled)
